(19) United States
(12) Patent Application Publication
(10) Pub. No.: US 2026/0057961 A1
(43) Pub. Date: Feb. 26, 2026

(54) CHROMOSOME 9P24 LOSS AND GAIN PREDICTS RESISTANCE AND BENEFIT OF IMMUNE CHECKPOINT INHIBITORS

(71) Applicants: THE REGENTS OF THE UNIVERSITY OF CALIFORNIA, Oakland, CA (US); NEW YORK UNIVERSITY, New York, NY (US)

(72) Inventors: Scott M. Lippman, La Jolla, CA (US); Webster K. Cavenee, Del Mar, CA (US); Teresa Davoli, New York, NY (US); Xin Zhao, New York, NY (US)

(21) Appl. No.: 19/104,197

(22) PCT Filed: Aug. 15, 2023
(86) PCT No.: PCT/US2023/030298

§ 371 Date: Feb. 14, 2025

Related U.S. Application Data
(60) Provisional application No. 63/398,500, filed on Aug. 16, 2022.
(60) Provisional application No. 63/411,498, filed on Sep. 29, 2022.

(51) Int. Cl. G16B 20/00; G16B 25/10

(52) U.S. Cl. CPC G16B 20/00; G16B 25/10

(57) ABSTRACT

The disclosure further provides diagnostic, prognostic, and therapeutic methods, which are based, at least in part, on determination of the identity of a genotype of interest identified herein.

### CROSS-REFERENCE TO RELATED APPLICATIONS

This application claims priority under 35 U.S.C. § 119 (e) to U.S. Provisional Application Nos. 63/411,498, filed Sep. 29, 2022 and 63/398,588; filed Aug. 16, 2022, the contents of which are incorporated herein by reference in their entireties.

### STATEMENT OF GOVERNMENT SUPPORT

This invention was made with government support under CA106451, CA097007, DE026644, and CA023100 awarded by the National Institutes of Health. The government has certain rights in the invention.

### BACKGROUND

Anti-PD1 immune-checkpoint therapy (ICT) therapy is an integral part of the standard of care in HNSC. The definitive demonstrations of improved efficacy came through randomized trials, initially in the recurrent/metastatic setting after platinum failure with anti-PD-1 antibodies nivolumab and pembrolizumab. These studies demonstrated improved overall survival with anti-PD-1 therapy compared to chemotherapy or cetuximab. Subsequently, KEYNOTE-048 tested either pembrolizumab monotherapy or pembrolizumab and chemotherapy against a triplet regimen of platinum/5FU/cetuximab in first-line recurrent/metastatic disease. This study demonstrated improved survival of pembrolizumab monotherapy in patients whose tumors expressed PD-L1 protein by immunohistochemistry. Despite remarkable deep and durable responses, the majority of patients do not benefit from anti-PD-1 therapy, even those whose tumors express high levels of PD-L1. Furthermore, in approximately 20% of patients with no PD-L1 expression treated with pembrolizumab alone, overall survival is worse compared to chemotherapy. It is clear that ICT-responsive tumors demonstrate evidence of an anti-tumor immune response likely related to local interferon-ψ (IFNψ) release-CD274 (which encodes PD-L1) is an IFNψ responsive gene. Evidence of this IFNψ anti-tumor immune response includes associations with CD8 T-cell infiltration, immune score, gene expression profiles, and PD-L1 protein expression. Although no predictive biomarker has been validated for HNSC, the latter is most widely used in clinical practice due to its simplicity and the fact that other assays have not proven to be more predictive. Genomic-based findings have been evaluated as candidate biomarkers of ICT benefits, orthogonal to biomarkers dependent on an IFNψ response. The most widely studied of these is tumor mutational burden, first reported to be elevated in HNSC, bladder and lung cancers. Pembrolizumab has been approved by the FDA for all cancers with a tumor mutational burden of ≥10 mutations/megabase, based on clinical trials with limited HNSC representation. Another tumor agnostic genomic biomarker that has garnered FDA approval for anti-PD1 antibodies are mismatch repair defects, rarely present in HNSC. Although immune-molecular studies have variably identified specific genomic/pathway alterations associated with resistance to ICT in diverse tumors and model systems, none are validated in HNSC for use in standard clinical practice. There is, therefore, an urgent unmet medical need for understanding mechanisms of resistance and improved predictive biomarkers to identify the patient subpopulation likely to respond to ICT, in order to optimize the likelihood of therapeutic success and reduce the immune-oncology (IO) adverse-event risks and expense of unnecessary treatment. This disclosure satisfies this need and provides related advantages as well.

### SUMMARY OF THE DISCLOSURE

Somatic copy-number alterations (SCNAs), notably losses containing interferons (IFNs) and IFN-pathway genes, many on chromosome 9p, predict immune-cold, immune-checkpoint therapy (ICT)-resistant tumors. Previously, 9p21.3 loss was found to be an early genetic driver of human papillomavirus-negative (HPV⁻) head and neck cancer (HNSC), associated with an immune-cold tumor-microenvironment (TME) signal, and recent evidence suggested that this immune-cold phenotype was greatly enhanced with large 9p-arm deletions, notably encompassing 9p24.1. In multi-omics, continuous-variable SCNA (including deep losses and high gains), TME (e.g., six CD8 T-cell level metrics) analyses of four HPV⁻ HNSC cohorts, Applicant found preferential 9p24-locus deletion to be a major effector of an immune-cold TME, driven by 9p24.1-band loss (CD8 T-cell reduction, p=0.03), and in turn by an essential telomeric immune-regulatory element-JAK2-CD274 (CD8 depletion, p=2.0E−3). In contrast, same genetic-region gains were immune-hot, ICT responsive. Applicant tested the 9p-alteration influence on survival, coincident with TME patterns, using whole-transcriptome data from an HPV⁻ HNSC cohort treated with anti-PD-1 antibodies or chemotherapy. There were inherent band-level ICT-survival differences, where 9p24.1 loss/gain dosage patterns, which correlated with copy-number changes, strongly predicted outcome. 9p21.3-immune associations were less prominent or non-existent. At a 9p24.1-expression threshold of 60th percentile, ICT-patient survival exceeded that of chemotherapy (p<0.01); whereas below this threshold, ICT survival was inferior to chemotherapy and to ICT-treated patients at (or above) this 60% threshold (p<0.01). These results remained significant in PD-L1-positive patients (PD-L1 CPS>=1). Such findings could be relevant to other (squamous) cancers, in which 9p24.1 gain/immune-hot associations exist.

Despite remarkable ICT advances for the most lethal, HPV⁻ subtype of HNSC, drug resistance remains prevalent, poorly understood, and largely unidentified by existing biomarker tests. SCNAs, including copy-number losses of interferons (IFNs) and IFN-pathway genes on chromosome 9p, correlate with immune-cold TMEs and/or ICT resistance; however, the genomic regions mediating these effects are unclear and likely tissue specific. Multiomic analyses of independent HPV⁻ HNSC cohorts identified preferential 9p24.1-immune-oncology (IO) associations: copy-number losses with immune-cold, ICT-resistance and gains with immune-hot, -responsive disease. At a 9p24.1-expression threshold of 60th percentile, ICT-median overall survival was 3-fold higher than that of chemotherapy; below this transcript threshold, ICT survival was inferior to chemotherapy. These 9p24.1-alteration/IO findings reveal genetically-defined ICT-sensitivity and -resistance in HPV⁻ squamous tumors.

Based on these findings, Applicant provides herein a method of treating cancer, comprising, or consisting essentially of, or yet further consisting of, measuring 9p gain in a sample isolated from a subject and administering a treatment for the cancer comprising at least one of Apitolisib, Torn 2, or GSK1059615 to the subject if the 9p gain of is detected in the sample. In another aspect, if 9p gain is not detected in the subject, at least one of Apitolisib, Torn 2, or GSK1059615 is not administered to the subject.

Further provided is a method of treating cancer, comprising, or consisting essentially of, or yet further consisting of, (a) providing a sample from a subject with cancer; (b) measuring 9p gain in the sample; and (c) administering a treatment for the cancer comprising at least one of Apitolisib, Torn 2, or GSK1059615 to the subject if the 9p gain of the sample is detected. In another aspect, if 9p gain is not detected in the subject, at least one of Apitolisib, Torn 2, or GSK1059615 is not administered to the subject.

For the purpose of these methods, the cancer can be any cancer, non-limiting examples of such comprises lung adenocarcinoma, lung squamous cell carcinoma, pancreatic adenocarcinoma, bladder urothelial carcinoma, skin cutaneous melanoma, ESCA-Squamous (esophageal carcinoma), ESCA-Adenocarcinoma (esophageal carcinoma), stomach adenocarcinoma, colorectal adenocarcinoma, cervical cancer, breast cancer, ovarian cancer, prostate cancer, sarcoma, or any combination thereof cancer. In one embodiment, the cancer comprises human papilloma virus negative head and neck cancer subject. The subject can be a mammal that is predisposed or subject to the cancer, for example a mammal, such as a canine or a human patient.

In one embodiment the 9p gain comprises a 9p24.1 or a 9p21.3 gain. In a further aspect, the measured 9p gain comprises a 9p24.1 expression threshold of at least a 60th percentile. In some cases, 9p gain may be measured by analyzing, reviewing, nucleic acid molecule (e.g., chromosome sequencing data) sequencing data of one or more patients and/or subjects. The sequencing data may be provided from a database e.g., the cancer genome atlas program (TCGA), Real World Cohort (RWC), Clinical Proteomic Tumor Analysis Consortium (CPTAC), or a combination thereof databases and/or data repositories. In some cases, the sequencing data may comprise whole transcriptome sequencing (WTS), whole exosome sequencing (WEST) data, or a combination thereof sequencing data of one or more patients and/or subjects. In some cases, the sequencing data may be obtained and/or provided from sequencing one or more biological samples of one or more patients and/or subjects. The sequencing may comprise next generation sequencing (NSG), long-read sequencing, or a combination thereof. In some cases, copy number of one or more genomic regions from the sequencing data may be determined and utilized by the methods, described elsewhere herein, to determine chromosome 9p gain and/or chromosome 9p loss. In some instances, copy number may comprise log base 2 of the copy number ratios.

Any appropriate sample can be used for the methods, non-limiting examples of such are provided herein. They can be from a tissue or tumor biopsy or a cell culture prepared from the subject's sample. Alternatively, the sample can be a purchased cell line for use in determining combination therapy. In one aspect, the sample comprises one or more cell lines. In one aspect, the cell line comprises at least 1, at least 2, at least 3, or at least 4 cell lines. In some cases, the cell lines may comprise cancerous cells from one or more cancers. In some cases, the one or more cancers may comprise lung squamous cell carcinoma (LUSC), lung adenocarcinoma (LUAD), pancreatic adenocarcinoma (PAAD), bladder cancer (BCLA), squamous esophageal cancer (ESCA), skin cutaneous melanoma (SKCM), stomach adenocarcinoma (STAD), COADREAD, or any combination thereof cancers.

The disclosed methods are in one suitable for determining personalized therapy for the subject.

Any appropriate method to determine 9p gain in the sample can be used, several of which are described herein. Non-limiting examples of such comprise, or consist essentially of, or consist of chromosomal microarrays configured to detect 9p gain of the sample, target panel sequencing of the sample, sequencing the sample's exome, sequencing the sample's whole genome, using a trained predictive model to analyze a structure of the sample stained with hematoxylin and eosin, or any combination thereof. In one aspect, the trained predictive model is trained with a plurality of chromosome 9p classification features and optionally wherein the sample's exome, whole genome, any fraction thereof, or any combination thereof are obtained from a publicly available database. In another aspect, the publicly available database comprises Genomic of Drug Sensitivity in Cancer (GDSC), Cancer Cell Line Encyclopedia (CCLE), cancer genome atlas program (TCGA), Real World Cohort (RWC), Clinical Proteomic Tumor Analysis Consortium (CPTAC), or any combination thereof.

As is apparent to the skilled artisan, the disclosed methods can be combined with other appropriate therapies, e.g., tumor resection, or other therapy and can be a first-line, second-line, third-line, fourth-line or fifth-line therapy. The cancer can be a primary tumor or cancer or metastatic cancer or tumor.

In one aspect, the treatment comprises, or consists essentially of, or consists of at least about 10, at least about 20, at least about 23, or at least about 30-fold increase in IC50 value when administered to the subject with the 9p gain in the sample.

In a further aspect, the treatment provides the subject a three-fold survival compared to the subject receiving chemotherapy e.g., cetuximab and/or platinum-based chemotherapies.

Methods for treating cancer in a subject are also provided herein. In one aspect, a method of treating cancer in a subject in need thereof is provided, the method comprising, or consisting essentially of, or consisting of: (a) providing a sample from a subject with cancer; (b) measuring 9p gain in the sample; and administering a treatment to the subject if the 9p gain of the sample is detected, wherein the treatment targets PI3k, Akt, mTOR, STING agonist, or any combination thereof signaling pathways. Also provided is a method of treating cancer in a subject in need thereof, the method comprising, or consisting essentially of, or yet further consisting of administering a treatment targeting at least one of Apitolisib, Torn 2, or GSK1059615 to the subject if the 9p gain of is detected in a sample isolated from the subject. The method can further comprise, or consist essentially of, or consist of measuring 9p gain in a sample isolated from a subject. In another aspect, if 9p gain is not detected in the subject from the subject, at least one of a treatment targeting Apitolisib, Torn 2, or GSK1059615 is not administered to the subject.

For the purpose of these methods, the cancer can be any cancer, non-limiting examples of such comprises lung adenocarcinoma, lung squamous cell carcinoma, pancreatic adenocarcinoma, bladder urothelial carcinoma, skin cutaneous melanoma, ESCA-Squamous (esophageal carcinoma), ESCA-Adenocarcinoma (esophageal carcinoma), stomach adenocarcinoma, colorectal adenocarcinoma, cervical cancer, breast cancer, ovarian cancer, prostate cancer, sarcoma, or any combination thereof cancer. In one embodiment, the cancer comprises human papilloma virus negative head and neck cancer subject. The subject can be a mammal that is predisposed or subject to the cancer, for example a mammal, such as a canine or a human patient.

In one embodiment the 9p gain comprises a 9p24.1 or a 9p21.3 gain. In a further aspect, the measured 9p gain comprises a 9p24.1 expression threshold of at least a 60th percentile. In some cases, 9p gain may be measured by analyzing, reviewing, nucleic acid molecule (e.g., chromosome sequencing data) sequencing data of one or more patients and/or subjects. The sequencing data may be provided from a database e.g., the cancer genome atlas program (TCGA), Real World Cohort (RWC), Clinical Proteomic Tumor Analysis Consortium (CPTAC), or a combination thereof databases and/or data repositories. In some cases, the sequencing data may comprise whole transcriptome sequencing (WTS), whole exosome sequencing (WEST) data, or a combination thereof sequencing data of one or more patients and/or subjects. In some cases, the sequencing data may be obtained and/or provided from sequencing one or more biological samples of one or more patients and/or subjects. The sequencing may comprise next generation sequencing (NSG), long-read sequencing, or a combination thereof. In some cases, copy number of one or more genomic regions from the sequencing data may be determined and utilized by the methods, described elsewhere herein, to determine chromosome 9p gain and/or chromosome 9p loss. In some instances, copy number may comprise log base 2 of the copy number ratios.

Any appropriate sample can be used for the methods, non-limiting examples of such are provided herein. They can be from a tissue or tumor biopsy or a cell culture prepared from the subject's sample. Alternatively, the sample can be a purchased cell line for use in determining combination therapy. In one aspect, the sample comprises one or more cell lines. In one aspect, the cell line comprises at least 1, at least 2, at least 3, or at least 4 cell lines. In some cases, the cell lines may comprise cancerous cells from one or more cancers. In some cases, the one or more cancers may comprise lung squamous cell carcinoma (LUSC), lung adenocarcinoma (LUAD), pancreatic adenocarcinoma (PAAD), bladder cancer (BCLA), squamous esophageal cancer (ESCA), SKCM, STAD, COADREAD, or any combination thereof cancers.

The disclosed methods are in one suitable for determining personalized therapy for the subject.

Any appropriate method to determine 9p gain in the sample can be used, several of which are described herein. Non-limiting examples of such comprise, or consist essentially of, or consist of chromosomal microarrays configured to detect 9p gain of the sample, target panel sequencing of the sample, sequencing the sample's exome, sequencing the sample's whole genome, using a trained predictive model to analyze a structure of the sample stained with hematoxylin and eosin, or any combination thereof. In one aspect, the trained predictive model is trained with a plurality of chromosome 9p classification features and optionally wherein the sample's exome, whole genome, any fraction thereof, or any combination thereof are obtained from a publicly available database. In another aspect, the publicly available database comprises Genomic of Drug Sensitivity in Cancer (GDSC), Cancer Cell Line Encyclopedia (CCLE), cancer genome atlas program (TCGA), Real World Cohort (RWC), Clinical Proteomic Tumor Analysis Consortium (CPTAC), or any combination thereof.

As is apparent to the skilled artisan, the disclosed methods can be combined with other appropriate therapies, e.g., tumor resection, or other therapy and can be a first-line, second-line, third-line, fourth-line or fifth-line therapy. The cancer can be a primary tumor or cancer or metastatic cancer or tumor.

In one aspect, the treatment comprises, or consists essentially of, or consists of at least about 10, at least about 20, at least about 23, or at least about 30-fold increase in IC50 value when administered to the subject with the 9p gain in the sample.

### BRIEF DESCRIPTION OF THE FIGURES

FIGS. 1A-1D: The association between SCNAs and immune infiltrates on HPV− HNSC. (FIG. 1A) Schematics of chromosome 9p, 9p24 and 9p21 loci, and the 3 bands at each locus. Different degrees of light shading (top 3 panels) indicate different bands on 9p24 locus (9p24.1, 9p24.2 and 9p24.3) with related example genes, and different degrees of darker shading (bottom 3 panels) indicate different bands intensity on 9p21 locus (9p21.1, 9p21.3 and 9p21.3) with related example genes on each cytoband. (FIG. 1B) Left panel: the percentage of patients with gains (light gray, above 0 line for patient percentage) or losses (darker gray, below 0 line for patient percentage) for HPV− HNSC for each chromosomal region. Each chromosome is split by a bold vertical dotted line, the p and q arms are split by the light vertical dotted line. The horizontal dotted line represents 10% and 20% of the patients. Right panel: The detailed plot on chromosome 9p only, black text with arrow marks the location of 9p24.1 and 9p21.3. The p and q arms are split by the light vertical dotted line, the horizontal dotted line represents the percentage of 10 and 20. (FIG. 1C) Correlation between SCNA (focal level, continuous variable) and CD8 T-cell level. Top Left panel: correlation between 9p arm-level SCNA and CD8 T-cell level, black dotted line indicated. Top right panel: correlation between 9p24.1 SCNA and CD8 T cells level; Bottom left panel: correlation between 9p21.3 focal SCNA and CD8 T cells level. Bottom right panel: correlation between JAK2-CD274 SCNA and CD8 T cells level. The cutoff of 50% copy number loss (vertical dashed line) or gain (for example 1 copy loss or gain for diploid genome, 1.5 copies loss or gain for triploid genome and 2 copies loss or gain for tetraploid genome) and the Spearman's correlation coefficients and related p-value are indicated in the top of the plot. (FIG. 1D) Area plot showing the association (Z-score) between gains (light gray) and losses (darker gray) and immune score (upper panel) or CD8 T-cell level (lower panel) across each chromosome. Each chromosome is split by a bold vertical dotted line, the p and q arms are split by the light vertical dotted line. The horizontal dot line represents p=0.05. Positive Z-score indicates the SCNA is positively associated with the immune score or CD8 T-cell level, negative Z-score indicates the SCNA is negatively associated with the immune score or CD8 T-cell level. The arrow above 9p indicates the location of 9p. The right high-power images are focused on the chromosome 9, text with black arrow indicates the location of 9p21.3 and 9p24.1. Light gray indicate gains and darker gray indicate losses.

FIGS. 2A-2C: The association between 9p and immune infiltrates on other solid-tumor types. (FIG. 2A) Area plot showing the percentage of patients and association with immune score for other cancer types (LUSC, LUAD, PAAD, ESCA-squamous, ESCA-adenocarcinoma, BLCA, SKCM, STAD and COADREAD) as indicated. Left panel represents the percentage of patients having SCNA on chromosome 9, the p and q arms are split by the light vertical dot line. The horizontal dot line represents 10% and 20% of the patients. Right panel represents the area plot showing the association (Z-score) between gains (light gray) and losses (darker gray) and immune score on chromosome 9, black text with arrow marks the location of 9p24.1 and 9p21.3. The p and q arms are split by the light vertical dot line. The horizontal dot line represents p=0.05. Positive Z-score indicates the SCNA is positively associated with the immune score, negative Z-score indicates the SCNA is negatively associated with the immune score. The text with black arrow indicates the location of 9p21.3 and 9p24.1. Light gray indicates gain and dark gray indicates loss. (FIG. 2B) Area plot showing the percentage of patients and association with immune score on chromosome 9 for squamous cancer types (HPV− HNSC, LUSC and ESCA-squamous), black text with arrow marks the location of 9p24.1 and 9p21.3. Left panel represents the percentage of patients having SCNA on chromosome 9, the p and q arms are split by the light vertical dot line. The horizontal dot line represents 10% and 20% of the patients. Right panel represents the area plot showing the association (Z-score) between gains (light gray) and losses (darker gray) and immune score on chromosome 9, black text with arrow marks the location of 9p24.1 and 9p21.3. The p and q arms are split by the light vertical dot line. The horizontal dot line represents p=0.05. Positive Z-score indicates the SCNA is positively associated with the immune score, negative Z-score indicates the SCNA is negatively associated with the immune score. The text with black arrow indicates the location of 9p21.3 and 9p24.1. Bottom panel represents the beta-coefficients and q-value from a multivariable logistic regression between immune score and sharp peak at 9p24.1 focal gain in squamous-cell cancers. (FIG. 2C) Area plot showing the association (Z-score) between gains (light gray) and losses (darker gray) and immune score on chromosome 9 for adenocarcinomas (LUAD, PAAD, STAD and ESCA-adenocarcinoma), black text with arrow marks the location of 9p24.1 and 9p21.3. Left panel represents the percentage of patients having SCNA on chromosome 9, the p and q arms are split by the light vertical dot line. The horizontal dot line represents 10% and 20% of the patients. Right panel represents the area plot showing the association (Z-score) between gains (light gray) and losses (dark gray) and immune score on chromosome 9, black text with arrow marks the location of 9p24.1 and 9p21.3. The p and q arms are split by the light vertical dot line. The horizontal dot line represents p=0.05. Positive Z-score indicates the SCNA is positively associated with the immune score, negative Z-score indicates the SCNA is negatively associated with the immune score. The text with black arrow indicates the location of 9p21.3 and 9p24.1. Light gray indicates gain and darker gray indicates loss. Bottom panel represents the beta-coefficients and q-value from a multivariable logistic regression between immune score and 9p24.1 focal gain in adenocarcinoma.

FIGS. 3A-3G: 9p24.1 transcriptome predicts better survival in HPV− HNSC. (FIG. 3A) Survival analysis of different expression percentile for different genes on 9p24.1. The histogram represents the q-value for each percentile. For example, for gene CD274, white histogram represents the survival difference between more than 20th percentile and less than 20^{th} percentile expression of CD274. Light gray histogram represents survival difference between more than 40^{th} percentile and less than 40^{th} percentile expression of CD274. Darker gray histogram represents survival difference between more than 60^{th} percentile and less than 60^{th} percentile expression of CD274. Dash line represents the significance of q-value=0.05, all the genes are sorted by genome coordinates. (FIG. 3B) Survival analysis of different expression percentiles for different genes on 9p21.3. The histogram represents the q-value for each percentile. For example, for gene CDKN2A, white histogram represents the survival difference between more than 20^{th} percentile less than 20^{th} percentile expression of CDKN2A. Light gray histogram represents survival difference between more than 40th percentile and less than 40^{th} percentile expression of CDKN2A. Darker gray histogram represents survival difference between more than 60^{th} percentile and less than 60^{th} percentile expression of CDKN2A. Dash line represents the significance of q-value-0.05, all the genes are sorted by genome coordinates. (FIG. 3C) HR plots for over- and under-expressors as a function of RNA expression percentile for JAK2-CD274 transcript correlate of 9p24.1. The maximum (peak) difference is at the 66^{th} percentile. At this optimal threshold, the maximal survival difference between the two cohorts indicates that there is a survival benefit exhibited by ICT-treated patients relative to non-ICT treated patients among those expressing this biomarker pair at this level. KMs for over/under at the 60^{th} percentile are shown below. For q-values in the ICT (IO) vs non-IO (ICT) groups are significant (q=0.019) for this biomarker. (FIG. 3D) Shows the change in hazard ratio (HR) for ICT vs non-ICT treated groups at each of the different percentile thresholds for 9p24.1 and 9p21.3. (FIG. 3E) Kaplan Meier curves for overall survival, measured from specimen collection date through last as a function of cumulative RNA expression based upon cohorts for patients above vs below the 20^{th}, 40^{th}, and 60^{th} percentile for 9p24.1 treated with and without IO. (FIG. 3F) Kaplan Meier curves for overall survival, measured from specimen collection date through last as a function of cumulative RNA expression based upon cohorts for patients above vs below the 60^{th} percentile for 9p24.1 treated with and without ICT (FIG. 3G) Kaplan Meier curves for overall survival as in (FIG. 3F) but in the PD-L1 IHC-positive subgroup only.

FIGS. 4A-4B: Correlation between SCNA (continuous variable) and immune infiltrates in HPV− HNSC (CPTAC) (FIG. 4A) Correlation between 9p24, 3 cytobands (9p24.1, 9p24.2, 9p24.3) focal-level SCNA and CD8 T cells. Both Spearman and Pearson's correlation coefficients and related p-value are indicated in the top of the plot. (FIG. 4B) Correlation between 9p24, 3 cytobands (9p24.1, 9p24.2, 9p24.3) focal-level SCNA and immune score. Both Spearman and Pearson's correlation coefficients and related p-value are indicated in the top of the plot.

FIGS. 5A-5B: Correlation between SCNA (continuous variable) and CD8 T cell enrichment in different TCGA cancer types (FIG. 5A) Correlation between 9p24.1 focal-level SCNA and CD8 T cell enrichment, black dot line indicated the cutoff of 50% copy number loss (for example, 1 copy loss for diploid genome, 1.5 copies loss for triploid genome and 2 copies loss for tetraploid genome). Spearman correlation coefficients and related p-value are indicated in the top of the plot. (FIG. 5B) Correlation between 9p21.3 focal-level SCNA and CD8 T cell enrichment, black dot line indicated the cutoff of 50% copy number loss (for example 1 copy loss for diploid genome, 1.5 copies loss for triploid genome and 2 copies loss for tetraploid genome). Spearman correlation coefficients and related p-value are indicated in the top of the plot.

FIGS. 6A-6B: Additional survival analysis for RWE cohort (FIG. 6A) Median overall survival increase is plotted as a function of expression (moving average of 2% bin size) of all patients independent of treatment or 9p-band status. Survival increase for patient cohorts defined by 9p24.1 expression percentile for patients treated with IO (gray line) or patients treated with any except IO (black line). Note the peak overall survival at 60 percentile, which could represent small patient numbers in the highest percentiles or a peak biologic effect at that threshold. Survival increase for patient cohorts defined by 9p21.3 expression percentiles for patients treated with IO (gray line) or patients treated with any except IO (black line). (FIG. 6B) Cox proportional hazard showing the relative risk at each expression percentile bin. There is a statistical significance to increasing expression of 9p21.3 on survival between the cohort treated with IO and those not treated with IO (p-value for expression and interaction <0.03). There is no statistical significance to increased or decreased expression of 9p21.3 on survival between the cohort treated with IO and those not treated with IO (p-values greater than 0.4). The greater the expression the better the survival for those treated with IO and at low expression, not being treated with IO confers a survival advantage.

FIG. 7: Loss frequency and association with immune score or CD8 T cells in HPV− HNSC and other TCGA cancers (SCNA were adjusted by ABSOLUTE).

FIG. 8: Association between CD8 T cells and SCNA in different methods.

FIG. 9: Association between immune parameters and copy number alterations in different cancer types.

FIG. 10: Spearman (Pearson) correlation analysis between CD8 and different cytobands.

FIG. 11: Association between CD8+ T cell and copy number alterations after considering deep loss and shallow loss.

### DETAILED DESCRIPTION

### Definitions

Throughout this disclosure, various publications, patents and published patent specifications are referenced by an identifying citation or by an Arabic numeral, the full citations for which are found immediately preceding the claims. The disclosures of these publications, patents and published patent specifications are hereby incorporated by reference into the present disclosure to more fully describe the state of the art to which this disclosure pertains.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

As used herein, the term “comprising” is intended to mean that the methods include the recited elements, but not excluding others. “Consisting essentially of” when used to define methods, shall mean excluding other elements of any essential significance to the method. “Consisting of” shall mean excluding more than trace elements of other ingredients for claimed compositions and substantial method steps. Embodiments defined by each of these transition terms are within the scope of this disclosure. Accordingly, it is intended that the methods can include additional steps and components (comprising) or alternatively including steps of no significance (consisting essentially of) or alternatively, intending only the stated method steps (consisting of).

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a cancer.

As used herein, the term “sample isolated from a subject” or “test sample” refers to any liquid or solid material containing nucleic acids. In suitable embodiments, a test sample is obtained from a biological source (i.e., a “biological sample”), such as cells in culture or a tissue sample from an animal, preferably, a human. In some embodiments, a biological sample comprises a sample selected from blood, serum, plasma, a throat swab, a nasal swab, a nasopharyngeal wash, saliva, urine, gastric fluid, cerebrospinal fluid, tears, stool, mucus, sweat, earwax, oil, a glandular secretion, semen, vaginal fluid, interstitial fluids derived from tumorous tissue, ocular fluids, breath, hair, finger nails, skin, biopsy tissue, placental fluid, amniotic fluid, cord blood, lymphatic fluids, cavity fluids, sputum, pus, microbiota, meconium, breast milk, and other secretions or excretions. In a specific embodiment, the sample is a biopsy sample.

The term “determining” or “identifying” is to associate or affiliate a patient closely to a group or population of patients who likely experience the same or a similar clinical response to a therapy.

As used herein, the term “deletion or loss of a genomic region” as referred to in a context of 9p, any 9p cytoband (e.g. 9p21, 9p24.1, 9p24, 9p21.3, 9p22, 9p13, CD274+JAK2) or gene (e.g. MLLT3, ELAVL2, KLH9, JAK2, CD274; or any of the other 20 genes, or combinations of genes, located at 9p24.1) as the presence of a genomic (DNA) copy number loss of the genomic region, using a cutoff between −0.15 and −0.3 for the log 2FC (log 2 fold change, where the fold change is the ratio between the copy number of the genomic region and the copy number of the rest of the genome). For example, if the copy number of 9p is 1 and the copy number of the rest of the genome is 2, the log 2FC is-1 and 9p is considered lost (see also Methods: PMID: 33952700).

As used herein, the term “decrease or suppression in a gene level” intends the presence of a significant decrease in the RNA level of the gene (measured by RNA sequencing) assessed using the following method as compared to a normal or healthy level. In one aspect, this can be determined by splitting the samples into 2 groups, for example 9p loss and no 9p loss. DESeq2 (https://bioconductor.org/packages/release/bioc/vignettes/DESeq2/inst/doc/DESeq2.html) or compatible program is used to detect the gene-level differential expression comparing the 2 groups of samples. The deriving p-values are adjusted by Benjamin and Hochberg or equivalent method for FDR. In one aspect, a cutoff of 0.05 p-value or 0.1 FDR is used to consider a gene significantly differentially expressed. In the case of PD-L1 protein level, it is measured by immune histochemistry with antibodies 22C3 or 28-8 (see also Methods: PMID: 33952700).

As used herein, the term “decrease or suppression in a pathway level” intends the presence of a significant decrease in the average RNA levels of the genes involved in the pathway (measured by RNA sequencing) based on a method called Gene Set Enrichment Analysis (https://www.gsea-msigdb.org/gsea/index.jsp) or equivalent method. Using the method disclosed above for “decrease in gene level” is performed and then run GSEA using the following score as the input: sign (log 2FoldChange)*−log 10 (adjusted p-value) where the log 2FC and the p-value for each gene are derived from the DESeq2 output. In one aspect, a cutoff of 0.05 p-value or 0.1 FDR is used to consider a pathway significantly differentially expressed (see also Methods: PMID: 33952700).

The term “selecting” a patient for a therapy refers to making an indication that the selected patient is suitable for the therapy. Such an indication can be made in writing by, for instance, a handwritten prescription or a computerized report making the corresponding prescription or recommendation.

“Having the same cancer” is used when comparing one patient to another or alternatively, one patient population to another patient population. For example, the two patients or patient population will each have or be suffering from colon cancer.

A “normal cell corresponding to the tumor tissue type” refers to a normal cell from a same tissue type as the tumor tissue. A non-limiting example is a normal lung cell from a patient having lung tumor, or a normal colon cell from a patient having colon tumor.

“Detecting, measuring or assessing” as used herein refers to determining the presence of a nucleic acid or gene of interest in a sample or the presence of a protein of interest in a sample. Detection does not require the method to provide 100% sensitivity and/or 100% specificity.

“Detectable label” as used herein refers to a molecule or a compound or a group of molecules or a group of compounds used to identify a nucleic acid or protein of interest. In some cases, the detectable label can be detected directly. In other cases, the detectable label can be a part of a binding pair, which can then be subsequently detected. Signals from the detectable label can be detected by various means and will depend on the nature of the detectable label. Detectable labels can be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluorescence, or chemiluminescence, or any other appropriate means.

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

When a genetic marker, e.g., SCNA, is used as a basis for selecting a patient for a treatment described herein, the genetic marker is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker in a clinical setting is a clear indication that this parameter was used as a basis for initiating, continuing, adjusting and/or ceasing administration of the treatments described herein.

### Cancer, Staging and Types

In certain embodiments, the terms “disease” “disorder” and “condition” are used interchangeably herein, referring to a precancer or alternatively cancer, a status of being diagnosed with a cancer, or a status of being suspect of having a cancer. “Cancer”, which is also referred to herein as “tumor”, is a known medically as an uncontrolled division of abnormal cells in a part of the body, benign or malignant. Non-limiting examples of malignant neoplasms include a broad group of diseases involving unregulated cell division and growth, and invasion to nearby parts of the body. Non-limiting examples of cancers include carcinomas, sarcomas, leukemia, and lymphoma, e.g., colon cancer, colorectal cancer, rectal cancer, gastric cancer, melanoma, non-small cell lung cancer, small cell lung cancer, esophageal cancer, head and neck cancer, HPV negative head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. In aspect, the cancer or precancer is not renal cancer.

Precancer cells or tumors are cells or tissue that contain abnormal cells that have an increased risk of turning cancerous.

Staging is the process of determining details about your cancer, such as tumor size and if it has spread. Typically, the stage guides treatment decisions. Stage I means the cancer is localized to the tissue where it originated. Stage II and III mean the cancer is larger and has grown into nearby tissues or lymph nodes. Stage IV indicates that the cancer cells are found in other organs from where the cancer originated.

In certain embodiments, the terms “disease” “disorder” and “condition” are used interchangeably herein, referring to a cancer, a status of being diagnosed with a cancer, or a status of being suspect of having a cancer. “Cancer”, which is also referred to herein as “tumor”, is a known medically as an uncontrolled division of abnormal cells in a part of the body, benign or malignant. In one embodiment, cancer refers to a malignant neoplasm, a broad group of diseases involving unregulated cell division and growth, and invasion to nearby parts of the body. Non-limiting examples of cancers include carcinomas, sarcomas, leukemia, and lymphoma, e.g., head and neck cancer, melanoma, colon cancer, colorectal cancer, rectal cancer, gastric cancer, esophageal cancer, head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. As used herein, an advanced cancer refers to a cancer that had progressed after receiving one or more of: the first line therapy, the second line therapy, or the third line therapy.

Head and neck cancer (HNC) develops from tissues in the lip and oral cavity (mouth), the larynx (throat), salivary glands, nose, sinuses or the skin of the face. The most common types of head and neck cancers occur in the lip, mouth, and larynx. HNC may be associated with prior infection with high-risk types of HPV (e.g., HPV-16 and -18) and is responsible for HPV-positive HNC. HPV positive and negative tumors represent a different clinicopathological and molecular entities. Many HPV-negative HNC are tobacco and alcohol inducted and are characterized by TP53 mutation. See http://atlasgeneticsoncology.org/Tumors/HeadNeckSCCID5078.html, last accessed on Jan. 29, 2022.

### Cancer Therapies and Methods of Administration

The term “suitable for a therapy” or “suitably treated with a therapy” shall mean that the patient is likely to exhibit one or more desirable clinical outcomes as compared to patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide or alternatively, SCNA. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease-free survival. In further another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, can exhibit more than one more desirable clinical outcomes as compared to patients having the same disease and receiving the same therapy but not possessing the characteristic. As defined herein, the patient is considered suitable for the therapy. In another such aspect, a patient possessing a characteristic can exhibit one or more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

As used herein, the term “administration” and “administering” are used to mean introducing an agent into a subject. Routes of administration include, but are not limited to, oral (such as a tablet, capsule, or suspension), topical, transdermal, intranasal, vaginal, rectal, subcutaneous intravenous, intravenous, intraarterial, intramuscular, intraosseous, intraperitoneal, intraocular, subconjunctival, sub-Tenon's, intravitreal, retrobulbar, intracameral, intratumoral, epidural and intrathecal.

An “effective amount” is an amount sufficient to effect beneficial or desired results. An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

“Therapeutically effective amount” of a drug or an agent refers to an amount of the drug or the agent that is an amount sufficient to obtain a pharmacological response or alternatively, is an amount of the drug or agent that, when administered to a patient with a specified disorder or disease, is sufficient to have the intended effect, e.g., treatment, alleviation, amelioration, palliation, or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

When the disease is cancer, the following clinical endpoints are non-limiting examples of treatment: (1) elimination of a cancer in a subject or in a tissue/organ of the subject or in a cancer loci; (2) reduction in tumor burden (such as number of cancer cells, number of cancer foci, number of cancer cells in a foci, size of a solid cancer, concentrate of a liquid cancer in the body fluid, and/or amount of cancer in the body); (3) stabilizing or delay or slowing or inhibition of cancer growth and/or development, including but not limited to, cancer cell growth and/or division, size growth of a solid tumor or a cancer loci, cancer progression, and/or metastasis (such as time to form a new metastasis, number of total metastases, size of a metastasis, as well as variety of the tissues/organs to house metastatic cells); (4) less risk of having a cancer growth and/or development; (5) inducing an immune response of the patient to the cancer, such as higher number of tumor-infiltrating immune cell, higher number of activated immune cells, or higher number cancer cell expressing an immunotherapy target, or higher level of expression of an immunotherapy target in a cancer cell; (6) higher probability of survival and/or increased duration of survival, such as increased overall survival (OS, which may be shown as 1-year, 2-year, 5-year, 10-year, or 20-year survival rate), increased progression free survival (PFS), increased disease free survival (DFS), increased time to tumor recurrence (TTR) and increased time to tumor progression (TTP). In some embodiments, the subject after treatment experiences one or more endpoints selected from tumor response, reduction in tumor size, reduction in tumor burden, increase in overall survival, increase in progression free survival, inhibiting metastasis, improvement of quality of life, minimization of drug-related toxicity, and avoidance of side-effects (e.g., decreased treatment emergent adverse events). In some embodiments, improvement of quality of life includes resolution or improvement of cancer-specific symptoms, such as but not limited to fatigue, pain, nausea/vomiting, lack of appetite, and constipation; improvement or maintenance of psychological well-being (e.g., degree of irritability, depression, memory loss, tension, and anxiety); improvement or maintenance of social well-being (e.g., decreased requirement for assistance with eating, dressing, or using the restroom; improvement or maintenance of ability to perform normal leisure activities, hobbies, or social activities; improvement or maintenance of relationships with family). In some embodiments, improved patient quality of life that is measured qualitatively through patient narratives or quantitatively using validated quality of life tools known to those skilled in the art, or a combination thereof. Additional non-limiting examples of endpoints include reduced hospital admissions, reduced drug use to treat side effects, longer periods off-treatment, and earlier return to work or caring responsibilities. In one aspect, prevention or prophylaxis is excluded from treatment.

Administration or treatment in “combination” refers to administering two agents such that their pharmacological effects are manifest at the same time. Combination does not require administration at the same time or substantially the same time, although combination can include such administrations.

The phrase “first line” or “second line” or “third line” etc., refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as primary therapy and primary treatment.” See National Cancer Institute website as www.cancer.gov, last visited on May 1, 2008. Typically, a patient is given a subsequent chemotherapy regimen because the patient did not shown a positive clinical or sub-clinical response to the first line therapy or the first line therapy has stopped.

The term “chemotherapy” or encompasses cancer therapies that employ chemical or biological agents or other therapies, such as radiation therapies, e.g., a small molecule drug or a large molecule, such as antibodies, Chimeric antigen receptor (CAR) therapies, RNAi and gene therapies. Non-limiting examples of chemotherapeutic agents are provided below. Unless specifically excluded, when a specific therapy is recited, equivalents of the therapy are within the scope of this disclosure.

An “immunotherapy agent” means a type of cancer treatment which uses a patient's own immune system to fight cancer, including but not limited to a physical intervene, a chemical substance, a biological molecule or particle, a cell, a tissue or organ, or any combinations thereof, enhancing or activating or initiating a patient's immune response against cancer. Non-limiting examples of immunotherapy agents include antibodies, immune regulators, checkpoint inhibitors, an antisense oligonucleotide (ASO), a RNA interference (RNAi), a Clustered Regularly Interspaced Short Palindromic Repeat (CRISPR) system, a viral vector, an anti-cancer cell therapy (e.g., transplanting an anti-cancer immune cell optionally amplified and/or activated in vivo, or administering an immune cell expressing a chimeric antigen receptor (CAR)), a CAR therapy, and cancer vaccines. As used herein, unless otherwise specified, an immunotherapy agent is not an inhibitor of thymidylate biosynthesis, or an anthracycline or other topoisomerase II inhibitor. As used herein, immune checkpoint refers to a regulator and/or modulator of the immune system (such as an immune response, an anti-tumor immune response, a nascent anti-tumor immune response, an anti-tumor immune cell response, an anti-tumor T cell response, and/or an antigen recognition of T cell receptor in the process of immune response). Their interaction activates either inhibitory or activating immune signaling pathways. Thus a checkpoint may contain one of the two signals: a stimulatory immune checkpoint that stimulates an immune response, and an inhibitory immune checkpoint inhibiting an immune response. In some embodiments, the immune checkpoint is crucial for self-tolerance, which prevents the immune system from attacking cells indiscriminately. However, some cancers can protect themselves from attack by stimulating immune checkpoint targets. In some embodiments, the immune checkpoints are present on T cells, antigen-presenting cells (APCs) and/or tumor cells.

One target of an immunotherapy agent is a tumor-specific antigen while the immunotherapy directs or enhances the immune system to recognize and attack tumor cells. Non-limiting examples of such agent includes a cancer vaccine presenting a tumor-specific antigen to the patient's immune system, a monoclonal antibody or an antibody-drug conjugate specifically binding to a tumor-specific antigen, a bispecific antibody specifically binding to a tumor-specific antigen and an immune cell (such as a T-cell engager or a NK-cell engager), an immune cell (such as a killer cell) specifically binding to a tumor-specific antigen (such as a CAR-T cell, a CAR-NK cell, and a CAR-NKT cell), a polynucleotide (or a vector comprising the same) transfecting/transducing an immune cell to express an tumor-specific antibody of an antigen binding fragment thereof (such as a CAR), or a polynucleotide (or a vector comprising the same) transfecting/transducing a cancer cell to express an antigen or a marker which can be recognized by an immune cell.

Another exemplified target is an inhibitory immune checkpoint which suppresses the nascent anti-tumor immune response, such as A2AR, B7-H3, B7-H4, BTLA, CTLA-4, CTLA-4/B7-1/B7-2, IDO, KIR, LAG3, NOX2, PD-1, PD-L1 and TIM-3, VISTA, SIGLEC7 (Sialic acid-binding immunoglobulin-type lectin 7, also designated as CD328) and SIGLEC9 (Sialic acid-binding immunoglobulin-type lectin 9, also designated as CD329). Non-limiting examples of such agent includes an antagonist or inhibitor of an inhibitory immune checkpoint, an agent reducing the expression and/or activity of an inhibitory immune checkpoint (such as via an antisense oligonucleotide (ASO), a RNA interference (RNAi), or a Clustered Regularly Interspaced Short Palindromic Repeat (CRISPR) system), an antibody or an antibody-drug conjugate or a ligand specifically binding to and reducing (or inhibiting) the activity of an inhibitory immune checkpoint, an immune cell with reduced (or inhibited) an inhibitory immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to reduce or inhibit an inhibitory immune checkpoint thereof. Reducing expression or activity of such inhibitory immune checkpoint enhances immune response of a patient to a cancer.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits, rat, canine, donkey, mice, camelids (such as dromedaries, llamas, and alpacas), as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, murine or humanized non-primate antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7^{th} Ed., W.H. Freeman & Co., 2013; Murphy, Janeway's Immunobiology, 8^{th} Ed., Garland Science, 2014; Male et al., Immunology (Roitt), 8^{th} Ed., Saunders, 2012; Parham, The Immune System, 4^{th} Ed., Garland Science, 2014. The term “antibody” includes any protein or peptide containing molecule that comprises at least a portion of an immunoglobulin molecule, such as the whole antibody and any antigen binding fragment or a single chain thereof. The terms “antibody,” “antibodies” and “immunoglobulin” also include immunoglobulins of any isotype, fragments of antibodies which retain specific binding to antigen, including, but not limited to, Fab, Fab′, F(ab)₂, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues. The antibodies can be polyclonal, monoclonal, multispecific (e.g., bispecific antibodies), and antibody fragments, so long as they exhibit the desired biological activity.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

In some embodiments, the antibody is a bispecific immune cell engager, referring to a bispecific monoclonal antibody that is capable of recognizing and specifically binding to a tumor antigen (such as CD19, EpCAM, MCSP, HER2, EGFR or CS-1) and an immune cell, and directing an immune cell to cancer cells, thereby treating a cancer. Non-limiting examples of such antibody include bispecific T cell engager, bispecific cytotoxic T lymphocytes (CTL) engager, and bispecific NK cell engager. In one embodiment, the engager is a fusion protein consisting of two single-chain variable fragments (scFvs) of different antibodies. Additionally, or alternatively, the immune cell is a killer cell, including but not limited to: a cytotoxic T cell, a gamma delta T cell, a NK cell and a NK-T cell.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells for using in a cell therapy and/or a CAR therapy may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses.

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells for using in a cell therapy and/or a CAR therapy may either be isolated or obtained from a commercially available source.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity or side effect.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at cancer.gov. Typically, a patient is given a subsequent therapy because the patient did not show a positive clinical or sub-clinical response to the first line therapy or the first line therapy has stopped.

The term “adjuvant” therapy refers to administration of a therapy or chemotherapeutic regimen to a patient in addition to the primary or initial treatment, such as after removal of a tumor by surgery. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure. Additionally, or alternatively, such adjuvant therapy potentials (i.e., sensitizes the subject to the original therapy) the subject may help reach one or more of clinical endpoints of the cancer treatment.

An “immunotherapy agent” means a type of cancer treatment which uses a patient's own immune system to fight cancer, including but not limited to a physical intervene, a chemical substance, a biological molecule or particle, a cell, a tissue or organ, or any combinations thereof, enhancing or activating or initiating a patient's immune response against cancer. Non-limiting examples of immunotherapy agents include antibodies, immune regulators, checkpoint inhibitors, an antisense oligonucleotide (ASO), a RNA interference (RNAi), a Clustered Regularly Interspaced Short Palindromic Repeat (CRISPR) system, a viral vector, an anti-cancer cell therapy (e.g., transplanting an anti-cancer immune cell optionally amplified and/or activated in vivo, or administering an immune cell expressing a chimeric antigen receptor (CAR)), a CAR therapy, and cancer vaccines. As used herein, unless otherwise specified, an immunotherapy agent is not an inhibitor of thymidylate biosynthesis, or an anthracycline or other topoisomerase II inhibitor. As used herein, immune checkpoint refers to a regulator and/or modulator of the immune system (such as an immune response, an anti-tumor immune response, a nascent anti-tumor immune response, an anti-tumor immune cell response, an anti-tumor T cell response, and/or an antigen recognition of T cell receptor in the process of immune response). Their interaction activates either inhibitory or activating immune signaling pathways. Thus, a checkpoint may contain one of the two signals: a stimulatory immune checkpoint that stimulates an immune response, and an inhibitory immune checkpoint inhibiting an immune response. In some embodiments, the immune checkpoint is crucial for self-tolerance, which prevents the immune system from attacking cells indiscriminately. However, some cancers can protect themselves from attack by stimulating immune checkpoint targets. In some embodiments, the immune checkpoints are present on T cells, antigen-presenting cells (APCs) and/or tumor cells.

One target of an immunotherapy agent is a tumor-specific antigen while the immunotherapy directs or enhances the immune system to recognize and attack tumor cells. Non-limiting examples of such agent includes a cancer vaccine presenting a tumor-specific antigen to the patient's immune system, a monoclonal antibody or an antibody-drug conjugate specifically binding to a tumor-specific antigen, a bispecific antibody specifically binding to a tumor-specific antigen and an immune cell (such as a T-cell engager or a NK-cell engager), an immune cell (such as a killer cell) specifically binding to a tumor-specific antigen (such as a CAR-T cell, a CAR-NK cell, and a CAR-NKT cell), a polynucleotide (or a vector comprising the same) transfecting/transducing an immune cell to express an tumor-specific antibody of an antigen binding fragment thereof (such as a CAR), or a polynucleotide (or a vector comprising the same) transfecting/transducing a cancer cell to express an antigen or a marker which can be recognized by an immune cell.

Another exemplified target is an inhibitory immune checkpoint which suppresses the nascent anti-tumor immune response, such as A2AR, B7-H3, B7-H4, BTLA, CTLA-4, CTLA-4/B7-1/B7-2, IDO, KIR, LAG3, NOX2, PD-1, PD-L1 and TIM-3, VISTA, SIGLEC7 (Sialic acid-binding immunoglobulin-type lectin 7, also designated as CD328) and SIGLEC9 (Sialic acid-binding immunoglobulin-type lectin 9, also designated as CD329). Non-limiting examples of such agent includes an antagonist or inhibitor of an inhibitory immune checkpoint, an agent reducing the expression and/or activity of an inhibitory immune checkpoint (such as via an antisense oligonucleotide (ASO), a RNA interference (RNAi), or a Clustered Regularly Interspaced Short Palindromic Repeat (CRISPR) system), an antibody or an antibody-drug conjugate or a ligand specifically binding to and reducing (or inhibiting) the activity of an inhibitory immune checkpoint, an immune cell with reduced (or inhibited) an inhibitory immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to reduce or inhibit an inhibitory immune checkpoint thereof. Reducing expression or activity of such inhibitory immune checkpoint enhances immune response of a patient to a cancer.

A further possible immunotherapy target is a stimulatory checkpoint molecule (including but not limited to 4-1BB, CD27, CD28, CD40, CD122, CD137, OX40, GITR and ICOS), wherein the immunotherapy agent actives or enhances the anti-tumor immune response. Non-limiting examples of such agent includes an agonist of a stimulatory checkpoint, an agent increasing the expression and/or activity of a stimulating immune checkpoint, an antibody or an antibody-drug conjugate or a ligand specifically binding to and activating or enhancing the activity of a stimulating immune checkpoint, an immune cell with increased expression and/or activity of a stimulating immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to express a stimulating immune checkpoint thereof.

As used herein the term “PD-1” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the PD-1 sequence as shown herein and/or a suitable binding partner of PD-L1. Non-limiting example sequences of PD-1 are provided herein, such as but not limited to those under the following reference numbers-GCID: GC02M241849; HGNC: 8760; Entrez Gene: 5133; Ensembl: ENSG00000188389; OMIM: 600244; and UniProtKB: Q15116—and the sequence: MQIPQAPWPVVWAVLQLGWRPGWFLDSPDRPWNPPTFSPALLVVTEGDNATFTCSF SNTSESFVLNWYRMSPSNQTDKLAAFPEDRSQPGQDCRFRVTQLPNGRDFHMSVVR ARRNDSGTYLCGAISLAPKAQIKESLRAELRVTERRAEVPTAHPSPSPRPAGQFQTLV VGVVGGLLGSLVLLVWVLAVICSRAARGTIGARRTGQPLKEDPSAVPVFSVDYGEL DFQWREKTPEPPVPCVPEQTEYATIVFPSGMGTSSPARRGSADGPRSAQPLRPEDGHC SWPL, and equivalents thereof. Non-limiting examples of commercially available antibodies thereto include pembrolizumab (Merck), nivolumab (Bristol-Myers Squibb), pidilizumab (Cure Tech), AMP-224 (GSK), AMP-514 (GSK), PDR001 (Novartis), and cemiplimab (Regeneron and Sanofi).

As used herein the term “PD-L1” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the PD-L1 sequence as shown herein and/or an suitable binding partner of PD-1. Non-limiting example sequences of PD-L1 are provided herein, such as but not limited to those under the following reference numbers-GCID: GC09P005450; HGNC: 17635; Entrez Gene: 29126; Ensembl: ENSG00000120217; OMIM: 605402; and UniProtKB: Q9NZQ7—and the sequence: MRIFAVFIFMTYWHLLNAFTVTVPKDLYVVEYGSNMTIECKFPVEKQLDLAALIVY WEMEDKNIIQFVHGEEDLKVQHSSYRQRARLLKDQLSLGNAALQITDVKLQDAGVY RCMISYGGADYKRITVKVNAPYNKINQRILVVDPVTSEHELTCQAEGYPKAEVIWTS SDHQVLSGKTTTTNSKREEKLFNVTSTLRINTTTNEIFYCTFRRLDPEENHTAELVIPE LPLAHPPNERTHLVILGAILLCLGVALTFIFRLRKGRMMDVKKCGIQDTNSKKQSDT HLEET, and equivalents thereof. Non-limiting examples of commercially available antibodies thereto include atezolizumab (Roche Genentech), avelumab (Merck Soreno and Pfizer), durvalumab (AstraZeneca), BMS-936559 (Bristol-Myers Suibb), and CK-301 (Checkpoint Therapeutics).

Additional or alternative targets may be utilized by an immunotherapy agent, such as an immune regulating agent, including but not limited to, an agent activating an immune cell, an agent recruiting an immune cell to a cancer or a cancer cell, or an agent increasing immune cell infiltrated into a solid tumor and/or a cancer loci. Non-limiting examples of such agent is an immune regulator or a variant, a mutant, a fragment, an equivalent thereof.

In some embodiments, an immunotherapy agent utilizes one or more targets, such as a bispecific T cell engager, a bispecific NK cell engager, or a CAR cell therapy. In some embodiments, the immunotherapy agent targets one or more immune regulatory or effector cells.

A “tumor response” (TR) refers to a tumor's response to therapy. A “complete response” (CR) to a therapy refers to the clinical status of a patient with evaluable but non-measurable disease, whose tumor and all evidence of disease have disappeared following administration of the therapy. In this context, a “partial response” (PR) refers to a response that is anything less than a complete response. “Stable disease” (SD) indicates that the patient is stable following the therapy. “Progressive disease” (PD) indicates that the tumor has grown (i.e., become larger) or spread (i.e., metastasized to another tissue or organ) or the overall cancer has gotten worse following the therapy. For example, tumor growth of more than 20 percent since the start of therapy typically indicates progressive disease. “Non-response” (NR) to a therapy refers to status of a patient whose tumor or evidence of disease has remained constant or has progressed.

“Overall Survival” (OS) refers to the length of time of a cancer patient remaining alive following a cancer therapy.

“Progression free survival” (PFS) or “Time to Tumor Progression” (TTP) refers to the length of time following a therapy, during which the tumor in a cancer patient does not grow. Progression-free survival includes the amount of time a patient has experienced a complete response, partial response, or stable disease.

“Disease free survival” refers to the length of time following a therapy, during which a cancer patient survives with no signs of the cancer or tumor.

“Time to Tumor Recurrence (TTR)” refers to the length of time, following a cancer therapy such as surgical resection or chemotherapy, until the tumor has reappeared (come back). The tumor may come back to the same place as the original (primary) tumor or to another place in the body.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

One chemotherapy is 5-Fluorouracil (5-FU) which belongs to the family of therapy drugs called pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluoroidine), 1-tetrahydrofuranyl-5-fluorouracil (ftorafur), capecitabine (Xeloda®), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamichael (1999) The Oncologist 4:478-487.

Cetuximab or Erbitux (commercially available from Lily) is an FDA-approved antibody to the epidermal growth factor receptor (EGFR) that is used alone or in combination with irinotecan (also known as CPT-11 or Camptosar) to treat various cancers. See https://chemocare.com/chemotherapy/drug-info/cetuximab.aspx.

Another chemotherapy is 5-FU based adjuvant therapy which refers to 5-FU alone or alternatively the combination of 5-FU with one or more other treatments, that include, but are not limited to radiation, methyl-CCNU, leucovorin, oxaliplatin (such as cisplatin), irinotecan, mitomycin, cytarabine, doxorubicin, cyclophosphamide, and levamisole, as well as an immunotherapy. Specific treatment adjuvant regimens are known in the art such as weekly Fluorouracil/Leucovorin, weekly Fluorouracil/Leucovorin+Bevacizumab, FOLFOX, FOLFOX-4, FOLFOX6, modified FOLFOX6 (mFOLFOX6), FOLFOX6 with bevacizumab, mFOLFOX6+Cetuximab, mFOLFOX6+Panitumumab, modified FOLFOX7 (mFOLFOX7), FOLFIRI, FOLFIRI with Bevacizumab, FOLFIRI+Ziv-aflibercept, FOLFIRI with Cetuximab, FOLFIRI+Panitumumab, FOLFIRI+Ramucirumab, FOLFOXIRI, FOLFIRI with FOLFOX6, FOLFOXIRI+Bevacizumab, FOLFOXIRI+Cetuximab, FOLFOXIRI+Panitumumab, Roswell Park Fluorouracil/Leucovorin, Roswell Park Fluorouracil/Leucovorin+Bevacizumab, Simplified Biweekly Infusional Fluorouracil/Leucovorin, Simplified Biweekly Infusional Fluorouracil/Leucovorin+Bevacizumab, and MOF (semustine (methyl-CCNU), vincrisine (Oncovin®) and 5-FU). For a review of these therapies see Beaven and Goldberg (2006) Oncology 20 (5): 461-470 as well as www.cancertherapyadvisor.com/home/cancer-topics/gastrointestinal-cancers/gastrointestinal-cancers-treatment-regimens/colon-cancer-treatment-regimens/. Other chemotherapeutics can be added, e.g., oxaliplatin or irinotecan.

Capecitabine is chemotherapy that is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is a chemotherapy which is an adjuvant used in cancer therapy. It is used in synergistic combination with 5-FU to improve efficacy of the chemotherapeutic agent. Without being bound by theory, addition of Leucovorin is believed to enhance efficacy of 5-FU by inhibiting thymidylate synthase. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid N-[4-[(2-amino-5-formyl-1,4,5,6,7,8-hexahydro-4-oxo-6-pteridinyl)methyl]amino]benzoyl], calcium salt (1:1).

“Oxaliplatin” (Eloxatin) is a chemotherapy that is a platinum-based chemotherapy drug in the same family as cisplatin and carboplatin. It is typically administered in combination with fluorouracil and leucovorin in a combination known as FOLFOX for the treatment of colorectal cancer. Compared to cisplatin, the two amine groups are replaced by cyclohexyldiamine for improved antitumor activity. The chlorine ligands are replaced by the oxalato bidentate derived from oxalic acid in order to improve water solubility. Equivalents to Oxaliplatin are known in the art and include, but are not limited to cisplatin, carboplatin, aroplatin, lobaplatin, nedaplatin, and JM-216 (see Mckeage et al. (1997) J. Clin. Oncol. 201:1232-1237 and in general, Chemotherapy for Gynecological Neoplasm, Curr. Therapy and Novel Approaches, in the Series Basic and Clinical Oncology, Angioli et al. Eds., 2004).

“FOLFOX” is chemotherapy that is an abbreviation for a type of combination therapy that is used to treat cancer. This therapy includes leucovorin (“FOL”), 5-FU (“F”), and oxaliplatin (“OX”) and encompasses various regimens, such as FOLFOX-4, FOLFOX-6, modified FOLOX-6, and FOLFOX-7, which vary in doses and ways in which each of the three drugs are administered. “FOLFIRI” is an abbreviation for a type of combination therapy that is used treat cancer and comprises, or alternatively consists essentially of, or yet further consists of 5-FU, leucovorin, and irinotecan. Information regarding these treatments are available on the National Cancer Institute's web site, cancer.gov, last accessed on May 30, 2020 as well as www.cancertherapyadvisor.com/home/cancer-topics/gastrointestinal-cancers/gastrointestinal-cancers-treatment-regimens/colon-cancer-treatment-regimens/, last accessed on May 30, 2020.

Irinotecan (CPT-11) is a chemotherapy sold under the trade name of Camptosar. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

S-1 is a chemotherapy that consists of three agents (at a molar ratio of 1:0.4:1): tegafur, 5-chloro-2-4-dihydroxypyridine, and potassium oxonate.

An “antifolate” is a drug or biologic chemotherapy that impairs the function of folic acids, e.g., an antimetabolite agent that inhibits the use of a metabolite, i.e., another chemical that is part of normal metabolism. In cancer treatment, antimetabolites interfere with DNA production, thus cell division and growth of the tumor. Non-limiting examples of these agents are dihydrofolate reductase inhibitors, such as methotrexate, Aminopterin, and Pemetrexed; thymidylate synthase inhibitors, such as Raltitrexed or Pemetrexed; purine based, i.e. an adenosine deaminase inhibitor, such as Pentostatin, a thiopurine, such as Thioguanine and Mercaptopurine, a halogenated/ribonucleotide reductase inhibitor, such as Cladribine, Clofarabine, Fludarabine, or a guanine/guanosine: thiopurine, such as Thioguanine; or Pyrimidine based, i.e. cytosine/cytidine: hypomethylating agent, such as Azacitidine and Decitabine, a DNA polymerase inhibitor, such as Cytarabine, a ribonucleotide reductase inhibitor, such as Gemcitabine, or a thymine/thymidine: thymidylate synthase inhibitor, such as a Fluorouracil (5-FU).

### DESCRIPTIVE EMBODIMENTS

The disclosure further provides diagnostic, prognostic, and therapeutic methods, which are based, at least in part, on determination of the identity of a genotype of interest identified herein.

For example, information obtained using the diagnostic assays described herein is useful for determining if a subject is suitable for cancer treatment of a given type. Based on the prognostic information, a doctor can recommend a therapeutic protocol, useful for reducing the malignant mass or tumor in the patient or treat cancer in the individual.

A patient's likely clinical outcome following a clinical procedure such as a therapy or surgery can be expressed in relative terms. For example, a patient having a particular genotype or expression level can experience relatively longer overall survival than a patient or patients not having the genotype or expression level. The patient having the particular genotype or expression level, alternatively, can be considered as likely to survive. Similarly, a patient having a particular genotype or expression level can experience relatively longer progression free survival, or time to tumor progression, than a patient or patients not having the genotype or expression level. The patient having the particular genotype or expression level, alternatively, can be considered as not likely to suffer tumor progression. Further, a patient having a particular genotype or expression level can experience relatively shorter time to tumor recurrence than a patient or patients not having the genotype or expression level. The patient having the particular genotype or expression level, alternatively, can be considered as not likely to suffer tumor recurrence. Yet in another example, a patient having a particular genotype or expression level can experience relatively more complete response or partial response than a patient or patients not having the genotype or expression level. The patient having the particular genotype or expression level, alternatively, can be considered as likely to respond. Accordingly, a patient that is likely to survive, or not likely to suffer tumor progression, or not likely to suffer tumor recurrence, or likely to respond following a clinical procedure is considered suitable for the clinical procedure.

It is to be understood that information obtained using the diagnostic assays described herein can be used alone or in combination with other information, such as, but not limited to, genotypes or expression levels of other genes, clinical chemical parameters, histopathological parameters, or age, gender, and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis, or treatment.

The methods are useful in the assistance of an animal, a mammal or yet further a human patient. For the purpose of illustration only, a mammal includes but is not limited to a human, a simian, a murine, a bovine, an equine, a porcine or an ovine subject.

### Genetic Mutations and Detection

As used herein, “9p21.3” (3p14 or 17p13.1) loss means either focal or arm loss (i.e., deletion at 9p21.3 region could derive from arm or focal events), unless specifically qualified as “focal only.”

The term “somatic copy-number alteration” (SCNA) intends an alteration in a gene copy number acquired by a cell that can be passed to the progeny of the mutated cell in the course of division. SCNA can be determined by methods known in the art. Non-limiting examples of such include fluorescent in situ hybridization, comparative genomic hybridization, array comparative genomic hybridization, single nucleotide polymorphism (SNP) array, genomic sequencing, high resolution microarray, and karyotype analysis. In one aspect, the SCNA is determined using a method comprising, consisting essentially of, or yet further consisting of SNP array.

### Modes for Carrying Out the Disclosure

### Methods for Treatment

Applicant provides herein a method of treating cancer in a subject selected for the treatment, the method comprising, or consisting essentially of, or yet further consisting of, administering a treatment to the subject selected from at least one of Apitolisib, Torn 2, or GSK1059615, wherein the subject is selected for the treatment if 9p gain is detected in a sample isolated from the subject.

For the purpose of the method, the cancer can be any cancer, non-limiting examples of such comprises lung adenocarcinoma, lung squamous cell carcinoma, pancreatic adenocarcinoma, bladder urothelial carcinoma, skin cutaneous melanoma, ESCA-Squamous (esophageal carcinoma), ESCA-Adenocarcinoma (esophageal carcinoma), stomach adenocarcinoma, colorectal adenocarcinoma, cervical cancer, breast cancer, ovarian cancer, prostate cancer, sarcoma, or any combination thereof cancer. In one embodiment, the cancer comprises human papilloma virus negative head and neck cancer subject. As used herein, the term “human papilloma virus negative head and neck cancer” intends head and neck cancers that are primarily associated with tobacco-derived carcinogens, excessive alcohol consumption, or both, and are collectively referred to as HPV-negative HNSCC. The subject can be a mammal that is predisposed or subject to the cancer, for example a mammal, such as a canine or a human patient.

In one embodiment the 9p gain comprises a 9p24.1 or a 9p21.3 gain. In a further aspect, the measured 9p gain comprises a 9p24.1 expression threshold of at least a 60^{th} percentile. In some cases, 9p gain may be measured by analyzing, reviewing, nucleic acid molecule (e.g., chromosome sequencing data) sequencing data of one or more patients and/or subjects. The sequencing data may be provided from a database e.g., the cancer genome atlas program (TCGA), Real World Cohort (RWC), Clinical Proteomic Tumor Analysis Consortium (CPTAC), or a combination thereof databases and/or data repositories. In some cases, the sequencing data may comprise whole transcriptome sequencing (WTS), whole exosome sequencing (WEST) data, or a combination thereof sequencing data of one or more patients and/or subjects. In some cases, the sequencing data may be obtained and/or provided from sequencing one or more biological samples of one or more patients and/or subjects. The sequencing may comprise next generation sequencing (NSG), long-read sequencing, or a combination thereof. In some cases, copy number of one or more genomic regions from the sequencing data may be determined and utilized by the methods, described elsewhere herein, to determine chromosome 9p gain and/or chromosome 9p loss. In some instances, copy number may comprise log base 2 of the copy number ratios.

Any appropriate sample can be used for the methods, non-limiting examples of such are provided herein. They can be from a tissue or tumor biopsy or a cell culture prepared from the subject's sample. Alternatively, the sample can be a purchased cell line for use in determining combination therapy. In one aspect, the sample comprises one or more cell lines. In one aspect, the cell line comprises at least 1, at least 2, at least 3, or at least 4 cell lines. In some cases, the cell lines may comprise cancerous cells from one or more cancers. In some cases, the one or more cancers may comprise lung squamous cell carcinoma (LUSC), lung adenocarcinoma (LUAD), pancreatic adenocarcinoma (PAAD), bladder cancer (BCLA), squamous esophageal cancer (ESCA), SKCM, STAD, COADREAD, or any combination thereof cancers.

Any appropriate method to determine 9p gain in the sample can be used, several of which are described herein. Non-limiting examples of such comprise, or consist essentially of, or consist of chromosomal microarrays configured to detect 9p gain of the sample, target panel sequencing of the sample, sequencing the sample's exome, sequencing the sample's whole genome, using a trained predictive model to analyze a structure of the sample stained with hematoxylin and eosin, or any combination thereof. In one aspect, the trained predictive model is trained with a plurality of chromosome 9p classification features and optionally wherein the sample's exome, whole genome, any fraction thereof, or any combination thereof are obtained from a publicly available database. In another aspect, the publicly available database comprises Genomic of Drug Sensitivity in Cancer (GDSC), Cancer Cell Line Encyclopedia (CCLE), cancer genome atlas program (TCGA), Real World Cohort (RWC), Clinical Proteomic Tumor Analysis Consortium (CPTAC), or any combination thereof.

As is apparent to the skilled artisan, the disclosed methods can be combined with other appropriate therapies, e.g., tumor resection, or other therapy and can be a first-line, second-line, third-line, fourth-line or fifth-line therapy. The cancer can be a primary tumor or cancer or metastatic cancer or tumor.

In one aspect, the treatment comprises, or consists essentially of, or consists of at least about 10, at least about 20, at least about 23, or at least about 30-fold increase in IC50 value when administered to the subject with the 9p gain in the sample.

In a further aspect, the treatment provides the subject a three-fold survival compared to the subject receiving chemotherapy e.g., cetuximab and/or platinum-based chemotherapies.

Further provided is a method of treating cancer, comprising, or consisting essentially of, or yet further consisting of measuring 9p gain in the sample and administering a treatment for the cancer comprising at least one of Apitolisib, Torn 2, or GSK1059615 to the subject if 9p gain of the sample is detected in the sample. In another aspect, if 9p gain is not detected in the subject, at least one of Apitolisib, Torn 2, or GSK1059615 is not administered to the subject. In a further aspect, the method further comprises providing a sample from a subject with cancer for assaying the 9p gain in the sample.

For the purpose of these methods, the cancer can be any cancer, non-limiting examples of such comprises lung adenocarcinoma, lung squamous cell carcinoma, pancreatic adenocarcinoma, bladder urothelial carcinoma, skin cutaneous melanoma, ESCA-Squamous (esophageal carcinoma), ESCA-Adenocarcinoma (esophageal carcinoma), stomach adenocarcinoma, colorectal adenocarcinoma, cervical cancer, breast cancer, ovarian cancer, prostate cancer, sarcoma, or any combination thereof cancer. In one embodiment, the cancer comprises human papilloma virus negative head and neck cancer subject. The subject can be a mammal that is predisposed or subject to the cancer, for example a mammal, such as a canine or a human patient.

In one embodiment the 9p gain comprises a 9p24.1 or a 9p21.3 gain. In a further aspect, the measured 9p gain comprises a 9p24.1 expression threshold of at least a 60^{th} percentile. In some cases, 9p gain may be measured by analyzing, reviewing, nucleic acid molecule (e.g., chromosome sequencing data) sequencing data of one or more patients and/or subjects. The sequencing data may be provided from a database e.g., the cancer genome atlas program (TCGA), Real World Cohort (RWC), Clinical Proteomic Tumor Analysis Consortium (CPTAC), or a combination thereof databases and/or data repositories. In some cases, the sequencing data may comprise whole transcriptome sequencing (WTS), whole exosome sequencing (WEST) data, or a combination thereof sequencing data of one or more patients and/or subjects. In some cases, the sequencing data may be obtained and/or provided from sequencing one or more biological samples of one or more patients and/or subjects. The sequencing may comprise next generation sequencing (NSG), long-read sequencing, or a combination thereof. In some cases, copy number of one or more genomic regions from the sequencing data may be determined and utilized by the methods, described elsewhere herein, to determine chromosome 9p gain and/or chromosome 9p loss. In some instances, copy number may comprise log base 2 of the copy number ratios

Any appropriate sample can be used for the methods, non-limiting examples of such are provided herein. They can be from a tissue or tumor biopsy or a cell culture prepared from the subject's sample. Alternatively, the sample can be a purchased cell line for use in determining combination therapy. In one aspect, the sample comprises one or more cell lines. In one aspect, the cell line comprises at least 1, at least 2, at least 3, or at least 4 cell lines. In some cases, the cell lines may comprise cancerous cells from one or more cancers. In some cases, the one or more cancers may comprise lung squamous cell carcinoma (LUSC), lung adenocarcinoma (LUAD), pancreatic adenocarcinoma (PAAD), bladder cancer (BCLA), squamous esophageal cancer (ESCA), skin cutaneous melanoma (SKCM), stomach adenocarcinoma (STAD), COADREAD, or any combination thereof cancers.

The disclosed methods are in one suitable for determining personalized therapy for the subject.

Any appropriate method to determine 9p gain in the sample can be used, several of which are described herein. Non-limiting examples of such comprise, or consist essentially of, or consist of chromosomal microarrays configured to detect 9p gain of the sample, target panel sequencing of the sample, sequencing the sample's exome, sequencing the sample's whole genome, using a trained predictive model to analyze a structure of the sample stained with hematoxylin and eosin, or any combination thereof. In one aspect, the trained predictive model is trained with a plurality of chromosome 9p classification features and optionally wherein the sample's exome, whole genome, any fraction thereof, or any combination thereof are obtained from a publicly available database. In another aspect, the publicly available database comprises Genomic of Drug Sensitivity in Cancer (GDSC), Cancer Cell Line Encyclopedia (CCLE), cancer genome atlas program (TCGA), Real World Cohort (RWC), Clinical Proteomic Tumor Analysis Consortium (CPTAC), or any combination thereof.

As is apparent to the skilled artisan, the disclosed methods can be combined with other appropriate therapies, e.g., tumor resection, or other therapy and can be a first-line, second-line, third-line, fourth-line or fifth-line therapy. The cancer can be a primary tumor or cancer or metastatic cancer or tumor.

In one aspect, the treatment comprises, or consists essentially of, or consists of at least about 10, at least about 20, at least about 23, or at least about 30-fold increase in IC50 value when administered to the subject with the 9p gain in the sample. In a further aspect, the treatment provides the subject a three-fold survival compared to the subject receiving chemotherapy e.g., cetuximab and/or platinum-based chemotherapies.

Methods for treating cancer in a subject are also provided herein. In one aspect, a method of treating cancer in a subject in need thereof is provided, the method comprising, or consisting essentially of, or consisting of: (a) providing a sample from a subject with cancer; (b) measuring 9p gain in the sample; and administering a treatment to the subject if the 9p gain of the sample is detected, wherein the treatment targets PI3k, Akt, mTOR, STING agonist, or any combination thereof signaling pathways. Also provided is a method of treating cancer in a subject in need thereof, the method comprising, or consisting essentially of, or yet further consisting of administering a treatment targeting at least one of Apitolisib, Torn 2, or GSK1059615 to the subject if the 9p gain of is detected in a sample isolated from the subject. The method can further comprise, or consist essentially of, or consist of measuring 9p gain in a sample isolated from a subject. In another aspect, if 9p gain is not detected in the subject from the subject, at least one of a treatment targeting Apitolisib, Torn 2, or GSK1059615 is not administered to the subject.

For the purpose of these methods, the cancer can be any cancer, non-limiting examples of such comprises lung adenocarcinoma, lung squamous cell carcinoma, pancreatic adenocarcinoma, bladder urothelial carcinoma, skin cutaneous melanoma, ESCA-Squamous (esophageal carcinoma), ESCA-Adenocarcinoma (esophageal carcinoma), stomach adenocarcinoma, colorectal adenocarcinoma, cervical cancer, breast cancer, ovarian cancer, prostate cancer, sarcoma, or any combination thereof cancer. In one embodiment, the cancer comprises human papilloma virus negative head and neck cancer subject. The subject can be a mammal that is predisposed or subject to the cancer, for example a mammal, such as a canine or a human patient.

In one embodiment the 9p gain comprises a 9p24.1 or a 9p21.3 gain. In a further aspect, the measured 9p gain comprises a 9p24.1 expression threshold of at least a 60^{th} percentile. In some cases, 9p gain may be measured by analyzing, reviewing, nucleic acid molecule (e.g., chromosome sequencing data) sequencing data of one or more patients and/or subjects. The sequencing data may be provided from a database e.g., the cancer genome atlas program (TCGA), Real World Cohort (RWC), Clinical Proteomic Tumor Analysis Consortium (CPTAC), or a combination thereof databases and/or data repositories. In some cases, the sequencing data may comprise whole transcriptome sequencing (WTS), whole exosome sequencing (WEST) data, or a combination thereof sequencing data of one or more patients and/or subjects. In some cases, the sequencing data may be obtained and/or provided from sequencing one or more biological samples of one or more patients and/or subjects. The sequencing may comprise next generation sequencing (NSG), long-read sequencing, or a combination thereof. In some cases, copy number of one or more genomic regions from the sequencing data may be determined and utilized by the methods, described elsewhere herein, to determine chromosome 9p gain and/or chromosome 9p loss. In some instances, copy number may comprise log base 2 of the copy number ratios.

Any appropriate sample can be used for the methods, non-limiting examples of such are provided herein. They can be from a tumor biopsy or a cell culture prepared from the subject's sample. Alternatively, the sample can be a purchased cell line for use in determining combination therapy. In one aspect, the sample comprises one or more cell lines. In one aspect, the cell line comprises at least 1, at least 2, at least 3, or at least 4 cell lines. In some cases, the cell lines may comprise cancerous cells from one or more cancers. In some cases, the one or more cancers may comprise lung squamous cell carcinoma (LUSC), lung adenocarcinoma (LUAD), pancreatic adenocarcinoma (PAAD), bladder cancer (BCLA), squamous esophageal cancer (ESCA), SKCM, STAD, COADREAD, or any combination thereof cancers.

The disclosed methods are in one suitable for determining personalized therapy for the subject.

Any appropriate method to determine 9p gain in the sample can be used, several of which are described herein. Non-limiting examples of such comprise, or consist essentially of, or consist of chromosomal microarrays configured to detect 9p gain of the sample, target panel sequencing of the sample, sequencing the sample's exome, sequencing the sample's whole genome, using a trained predictive model to analyze a structure of the sample stained with hematoxylin and eosin, or any combination thereof. In one aspect, the trained predictive model is trained with a plurality of chromosome 9p classification features and optionally wherein the sample's exome, whole genome, any fraction thereof, or any combination thereof are obtained from a publicly available database. In another aspect, the publicly available database comprises Genomic of Drug Sensitivity in Cancer (GDSC), Cancer Cell Line Encyclopedia (CCLE), cancer genome atlas program (TCGA), Real World Cohort (RWC), Clinical Proteomic Tumor Analysis Consortium (CPTAC), or any combination thereof.

As is apparent to the skilled artisan, the disclosed methods can be combined with other appropriate therapies, e.g., tumor resection, or other therapy and can be a first-line, second-line, third-line, fourth-line or fifth-line therapy. The cancer can be a primary tumor or cancer or metastatic cancer or tumor.

In one aspect, the treatment comprises, or consists essentially of, or consists of at least about 10, at least about 20, at least about 23, or at least about 30-fold increase in IC50 value when administered to the subject with the 9p gain in the sample.

### Administration and Dosing

The appropriate amount and dosing regimen of the active agent to be administered to the subject according to any of the methods disclosed herein, is determined by one of ordinary skill in the art. In some embodiments, the active agents, or salts or solvates thereof, is administered to a subject suffering from abnormal cell growth, such as a human, either alone or as part of a pharmaceutically acceptable formulation, once a week, once a day, twice a day, three times a day, or four times a day, or even more frequently.

Administration can be affected by any method that enables delivery of the compounds to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the chemotherapeutic or immunotherapeutic agent are well-known in the relevant art and would be understood to be encompassed by the skilled artisan once provided the teachings disclosed herein.

### EXPERIMENTAL METHODS

### Experiment No. 1

### Introduction

Anti-PD1 immune-checkpoint therapy (ICT) therapy is an integral part of the standard of care in HNSC (4). The definitive demonstrations of improved efficacy came through randomized trials, initially in the recurrent/metastatic setting after platinum failure with anti-PD-1 antibodies nivolumab and pembrolizumab (5, 6). These studies demonstrated improved overall survival with anti-PD-1 therapy compared to chemotherapy or cetuximab. Subsequently, KEYNOTE-048 tested either pembrolizumab monotherapy or pembrolizumab and chemotherapy against a triplet regimen of platinum/5FU/cetuximab in first-line recurrent/metastatic disease (7). This study demonstrated improved survival of pembrolizumab monotherapy in patients whose tumors expressed PD-L1 protein by immunohistochemistry. Despite remarkable deep and durable responses, the majority of patients do not benefit from anti-PD-1 therapy, even those whose tumors express high levels of PD-L1 (8). Furthermore, in approximately 20% of patients with no PD-L1 expression treated with pembrolizumab alone, overall survival is worse compared to chemotherapy (9). It is clear that ICT-responsive tumors demonstrate evidence of an anti-tumor immune response likely related to local interferon-γ (IFNγ) release-CD274 (which encodes PD-L1) is an IFNγ responsive gene. Evidence of this IFNγ anti-tumor immune response includes associations with CD8 T-cell infiltration, immune score, gene expression profiles, and PD-L1 protein expression (10). Although no predictive biomarker has been validated for HNSC, the latter is most widely used in clinical practice due to its simplicity and the fact that other assays have not proven to be more predictive. Genomic-based findings have been evaluated as candidate biomarkers of ICT benefits, orthogonal to biomarkers dependent on an IFNγ response. The most widely studied of these is tumor mutational burden, first reported to be elevated in HNSC, bladder and lung cancers (11). Pembrolizumab has been approved by the FDA for all cancers with a tumor mutational burden of >10 mutations/megabase, based on clinical trials with limited HNSC representation (12). Another tumor agnostic genomic biomarker that has garnered FDA approval for anti-PD1 antibodies are mismatch

repair defects (13, 14), rarely present in HNSC. Although immune-molecular studies have variably identified specific genomic/pathway alterations associated with resistance to ICT in diverse tumors and model systems (15-18), none are validated in HNSC for use in standard clinical practice. There is, therefore, an urgent unmet medical need for understanding mechanisms of resistance and improved predictive biomarkers to identify the patient subpopulation likely to respond to ICT, in order to optimize the likelihood of therapeutic success and reduce the immune-oncology (IO) adverse-event risks and expense of unnecessary treatment.

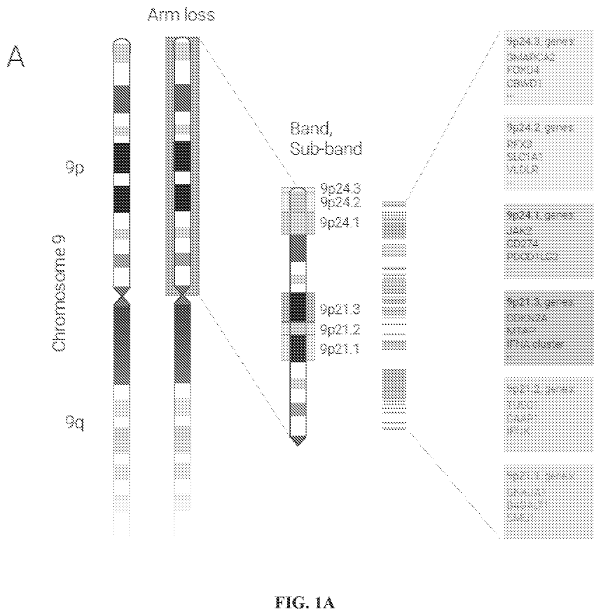

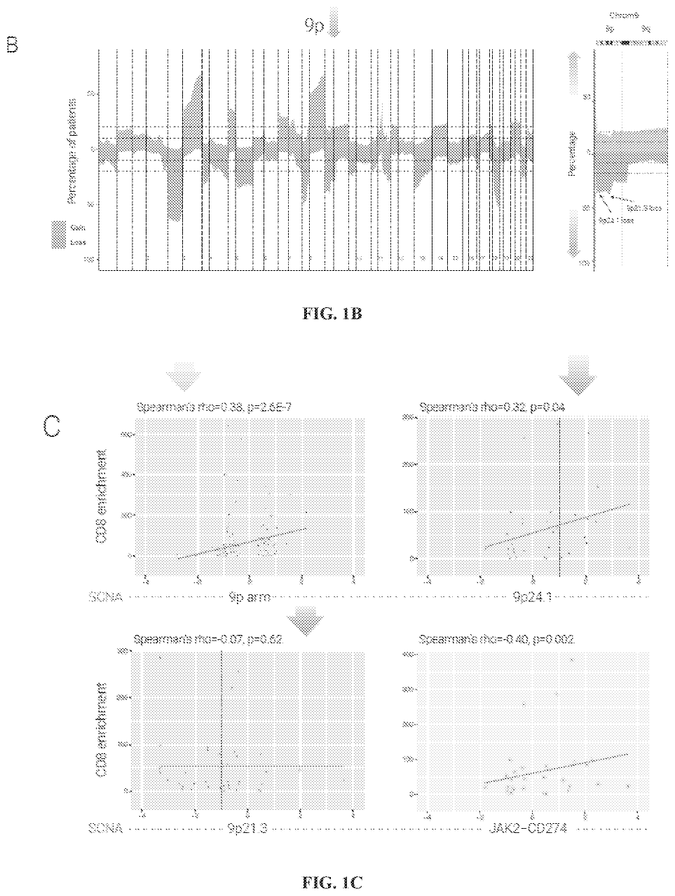

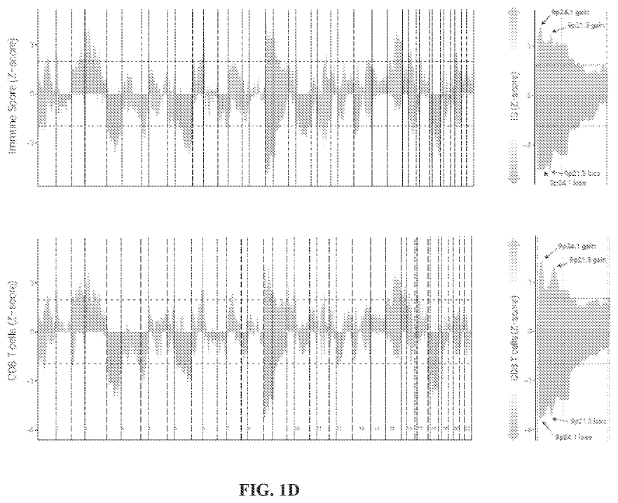

Somatic copy-number alterations (SCNAs), central chromosomal events in most cancers, can increase or decrease the dosage of specific genomic regions. Deletions of 9p21.3 (19) and 9p (20), among the most frequent recurrent SCNA events in human cancer, have been implicated in tumor initiation, evolution and progression through cell-cycle and tumor metabolism regulation. Studies of 9p21.3 copy-number alterations, primarily focused on CDKN2A/CDKN2B and MTAP deletions (21, 22), can encompass a cluster of 16 type-I interferon (IFN) genes (FIG. 1A) involved in anti-tumor immune responses (23); while IFN-pathway gene alterations at 9p24 have been reported to correlate with immune-cold, ICTresistant tumors, primarily in metastatic melanoma (24). Notably associated with 9p24.1-ICT resistance are loss-of-function mutations in JAK2, and IFN-γ resistance in cell lines lacking JAK2 (18). These findings were extended by reports of overall SCNA and copy-number loss (but not gain) burden, including 9p, associated with dual PD-1 and CTLA4 checkpoint-inhibitor resistant metastatic melanoma (1, 25).

Previously, Applicant identified recurrent 9p21.3 loss as an early genetic driver of human papillomavirusnegative (HPV−) HNSC (26), associated with an immune-cold signal limited to HPV− disease (2). Recent studies confirmed these 9p21.3 deletion/IO observations also limited to HPV− HNSC, including CD8 T-cell depletion and CXCL9 suppression, broadened these findings to several other solid tumors, and suggested that co-deletions extending to 9p24.1 were important to the immune-cold tumor microenvironments (TMEs) (3). These studies gave rise to new questions as to which genetic regions/genes on 9p are the main drivers of the immune-cold phenotype, along with the underlying molecular mechanism. The 9p24.1 region harbors the IFN-γ-related gene JAK2, as well as CD274 (encoding PD-L1) and PDCDILG2 (encoding PD-L2)—both ligands of PD-1 and targets of PD1/PD-L1 axis inhibitors (FIG. 1A), and whose co-deletions have not been fully characterized in immune oncology. In contrast, amplifications of genes at 9p24.1 have been associated with an increased abundance of the PD-1 ligands, PD-L1 and PD-L2, and exquisite ICT sensitivity, initially reported in classic Hodgkin's lymphoma, and recently in various solid tumors (27). Despite these emerging 9p-related immune gene and ICT effects, attempts to dissect 9p have failed to reveal a clear candidate mediator, likely to be tissue specific, of immune-response and ICT benefit. Here, Applicant extend the 9p/IO research to whole exome and -transcriptome, continuous-variable dissection of somatic alterations of 9p21.3, 9p24.1 or both (at locus, band and gene levels) in four HPV− HNSC cohorts, to computationally assess their copy number and transcript contributions to immune-cold/hot phenotypes and resistance/sensitivity to immunotherapy.

Somatic copy-number alterations (SCNAs), notably losses containing interferons (IFNs) and IFN-pathway genes, many on chromosome 9p, predict immune-cold, immune-checkpoint therapy (ICT)-resistant tumors (1). Previously, 9p21.3 loss was found to be an early genetic driver of human papilloma virus negative (HPV−) head and neck cancer (HNSC), associated with an immune-cold tumor microenvironment (TME) signal (2), and recent evidence suggested that this immune-cold phenotype was greatly enhanced with large 9p-arm deletions, notably encompassing 9p24.1 (3). In multi-omics, continuous-variable SCNA (including deep losses and high gains), TME (e.g., six CD8 T-cell level metrics) analyses of four HPV− HNSC cohorts, Applicant found preferential 9p24-locus deletion to be a major effector of an immune-cold TME, driven by 9p24.1-band loss (CD8 T-cell reduction, p=0.03), and in turn by an essential telomeric immune-regulatory element—JAK2-CD274 (CD8 depletion, p=2.0E−3). In contrast, same genetic-region gains were immune-hot, ICT responsive. Applicant then tested the 9p-alteration influence on survival, coincident with TME patterns, using whole-transcriptome data from an HPV− HNSC cohort treated with anti-PD-1 antibodies or chemotherapy. There were inherent band-level ICT-survival differences, where 9p24.1 loss/gain dosage patterns, which correlated with copy-number changes, strongly predicted outcome. 9p21.3-immune associations were less prominent or non-existent. At a 9p24.1-expression threshold of 60th percentile, ICT-patient survival exceeded that of chemotherapy (p<0.01); whereas below this threshold, ICT survival was inferior to chemotherapy and to ICT-treated patients at (or above) this 60% threshold (p<0.01). These results remained significant in PD-L1-positive patients (PD-L1 CPS >=1). Such findings could be relevant to other (squamous) cancers, in which 9p24.1 gain/immune-hot associations exist.

### Significance Statement

Despite remarkable ICT advances for the most lethal, HPV-subtype of HNSC, drug resistance remains prevalent, poorly understood, and largely unidentified by existing biomarker tests. SCNAs, including copy-number losses of interferons (IFNs) and IFN-pathway genes on chromosome 9p, correlate with immune-cold TMEs and/or ICT resistance; however, the genomic regions mediating these effects are unclear and likely tissue specific. Multiomic analyses of independent HPV− HNSC cohorts identified preferential 9p24.1-immune-oncology (IO) associations: copy-number losses with immune-cold, ICT resistance and gains with immune-hot, -responsive disease. At a 9p24.1-expression threshold of 60^{th} percentile, ICT-median overall survival was 3-fold higher than that of chemotherapy; below this transcript threshold, ICT survival was inferior to chemotherapy. These 9p24.1-alteration/IO findings reveal genetically-defined ICT-sensitivity and -resistance in HPV− squamous tumors.

### Results

9p24.1 SCNA is Associated with TME Phenotype in HPV− HNSC.

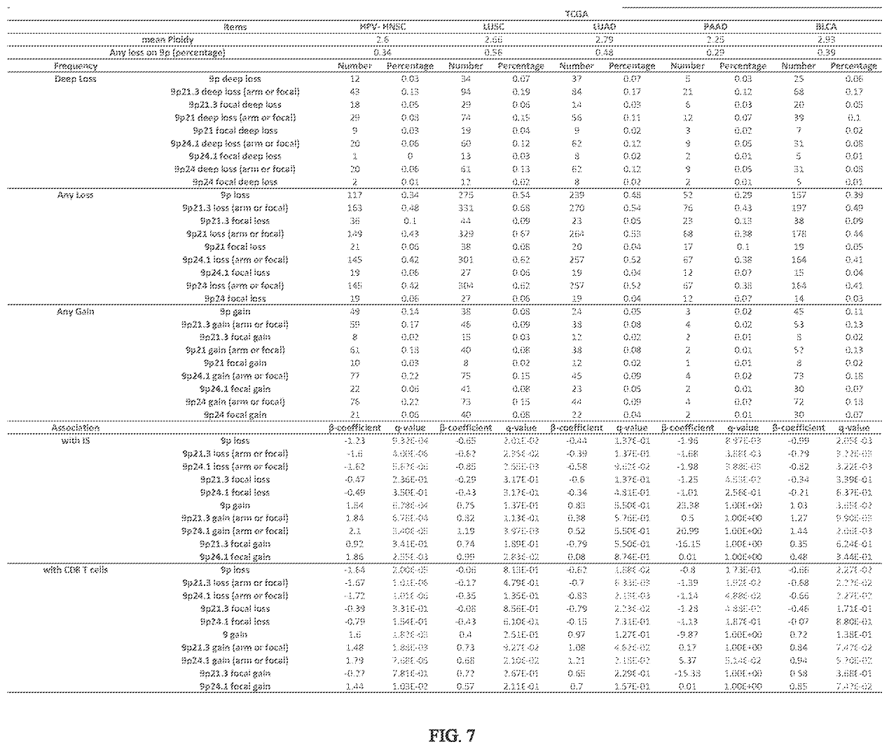

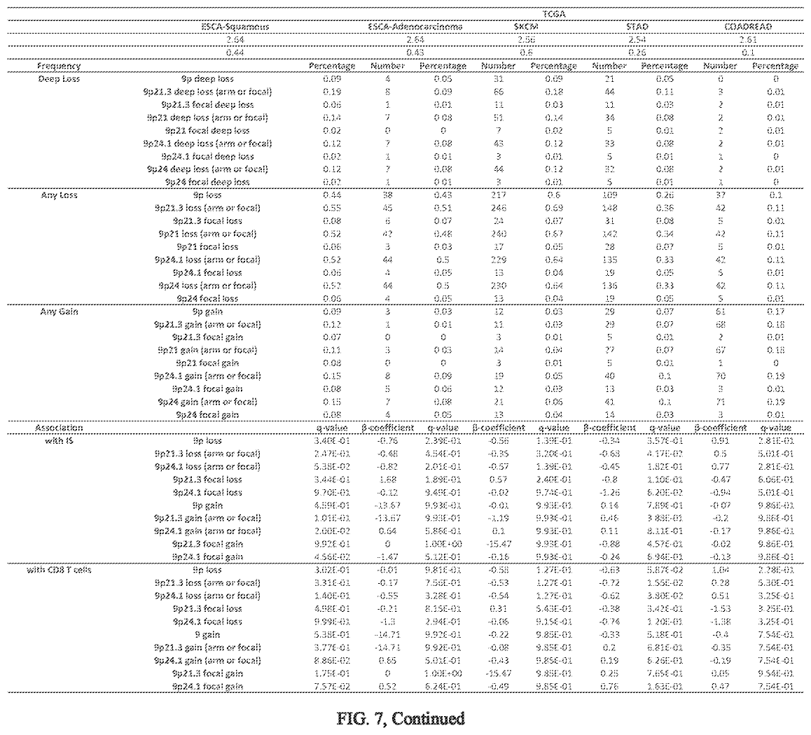

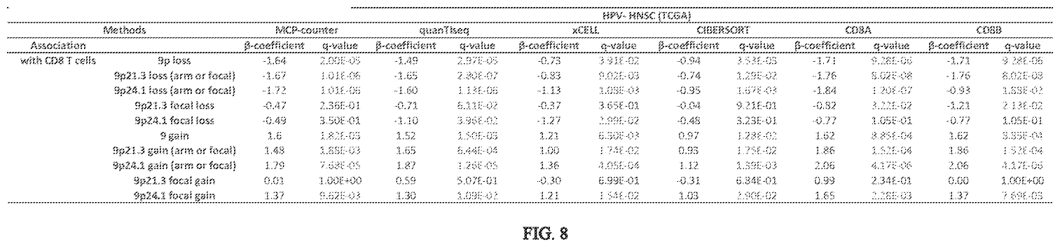

Among 343 HPV− HNSC patients with genomic SCNA data derived from TCGA, copy-number loss and gain frequencies for 9p arm, 9p21.3 and 9p24.1 were 34%, 48% and 42%, and 14%, 17% and 22%, respectively. In this analysis Applicant analyzed band loss at the ‘deep’ and homozygous-deletion levels, as recently reported (3, 21), as well as tumors with high-level (>2.5 copies) gains (FIGS. 1A-1B, FIG. 7). The results confirm the high rates of 9p21.3 band loss (19), but also reveal high rates of deep deletions in this band, controlling for tumor ploidy (more than 50% log 2 transformed copy-number loss). Applicant first studied the association between SCNA (gain or loss) events of each chromosomal region along the genome and the immune score or CD8 T-cell level using logistic regression after controlling for overall SCNA level. In brief, the cytotoxic immune score was based on the RNA expression of the cytotoxic markers GZMH, PRF1, CD3E, CD247, CD2, GZMK and NKG7 (2). CD8 T-cell levels were evaluated by MCPcounter (28), a deconvolutional method based on the normalized log 2-transformed gene expression matrix to infer the absolute abundance scores for CD8 T-cell level; and the results were validated by several other methods: quanTIseq (29), CIBERSORT (30), xCELL, (31), and CD8A and CD8B RNA expression (see Methods). Applicant used Z-score (FIG. 1D) for each chromosomal region to represent the association between immune infiltrates and SCNAs in the corresponding chromosomal regions (after controlling for the SCNA level), with a positive and negative Z-score indicating, respectively, positive and negative associations of SCNA events with immune infiltrates. The results for CD8 T-cell level associations were highly consistent with different methods and markers (FIG. 8). In addition, the variance analysis showed that gene-level SCNA on 9p24.1 or 9p21.3 across different patients had similar gain or loss trends (median variance=0.94, data not shown). In addition to negative associations of 9p loss (notably strong for 9p24.1) with immune score, with β (B-coefficient)=−1.23, q-value (FDR adjusted p-value)=9.3E−4, 9p-arm gain (peak at 9p24.1 shown by arrows in FIG. 1D, right side expanded images) had a similarly strong positive association with immune score and CD8 T-cell levels, with β=1.84, q-value=6.8E−4 (FIG. 7).

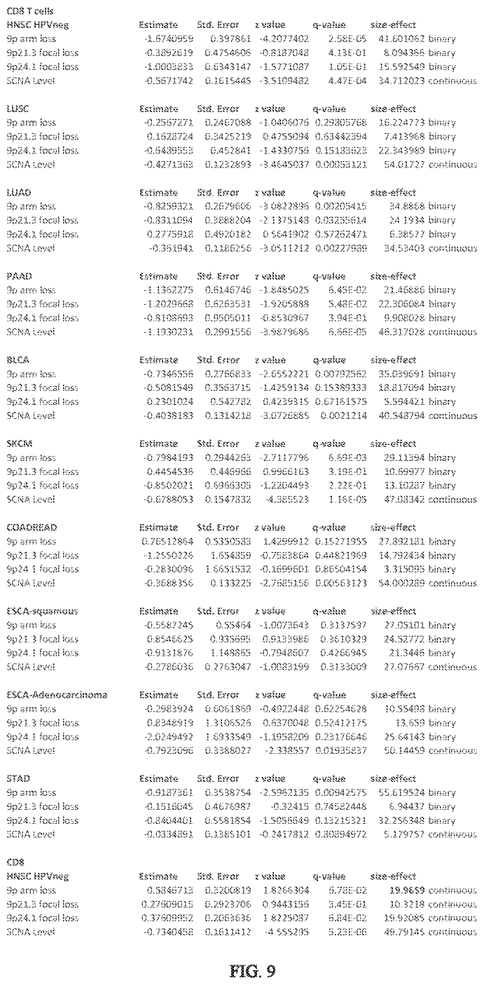

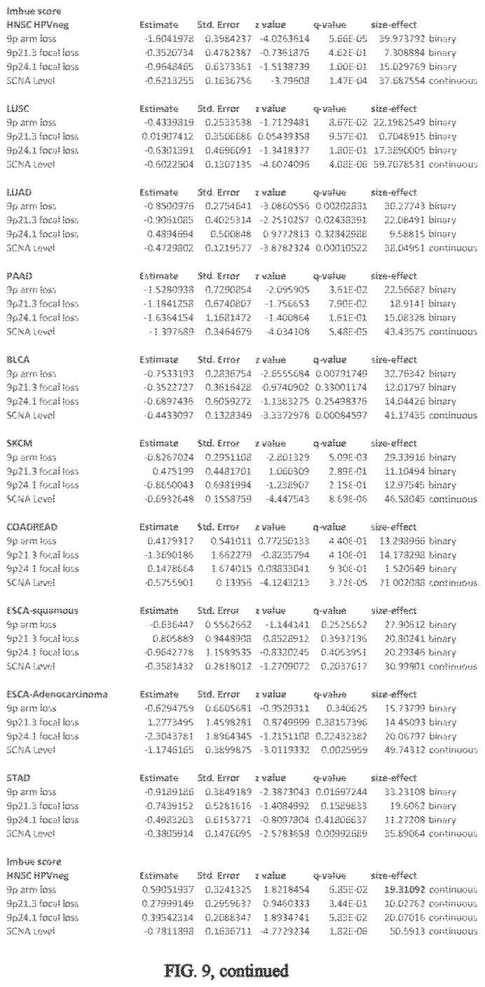

To assess the relative contributions of 9p21.3 and 9p24.1 loss to immune-cold TMEs, Applicant applied a logistic model to predict immune score or CD8 T-cell levels, again using focal events (excluding arm-level events). Consistent with previous studies and analyses above, both 9p-arm loss and SCNA levels were Zhao 9p24.1-p. 6 significant predictors of low immune score (9p arm loss: β=−1.60, q-value=5.7E−5; SCNA level: β=−0.62, qvalue=1.5E−4) and CD8 T-cell level (9p arm loss: γ=−1.67, q-value=2.6E−5; SCNA level: β=−0.57, qvalue=4.5E−4) (FIG. 9). Although limited by the small sample size (N=20), analysis of the 9p24.1, but not 9p21.3, loss subgroup showed a trend for the prediction of low immune score (9p24.1: β=−0.96, qvalue=0.10; 9p21.3: β=−0.39, q-value=0.41) and CD8 T-cell level (9p24.1: β=−1.00, q-value=0.11; 9p21.3: β=−0.35, q-value=0.46). The significance of 9p24.1 gain was observed when Applicant applied continuous instead of categorical SCNA values for the association (CD8 T cell: β-0.37, q-value-0.07; immune score: β=0.39, q-value=0.06). Size-effect (variable-importance) analysis showed that 9p loss could explain 42% of the variance for CD8 T-cell level and 40% for immune score (FIG. 9).

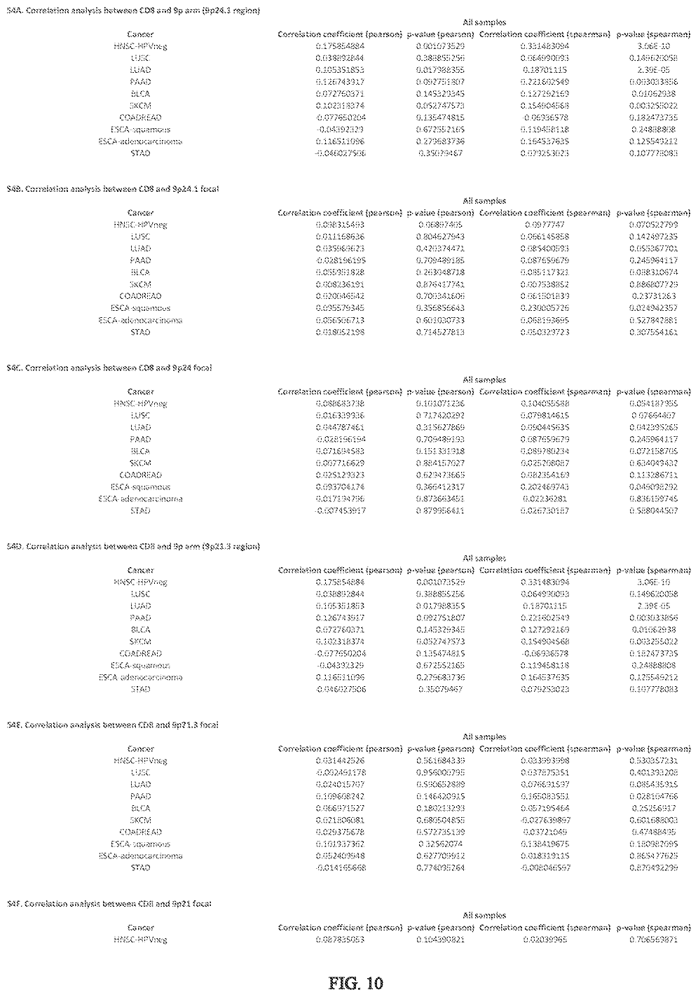

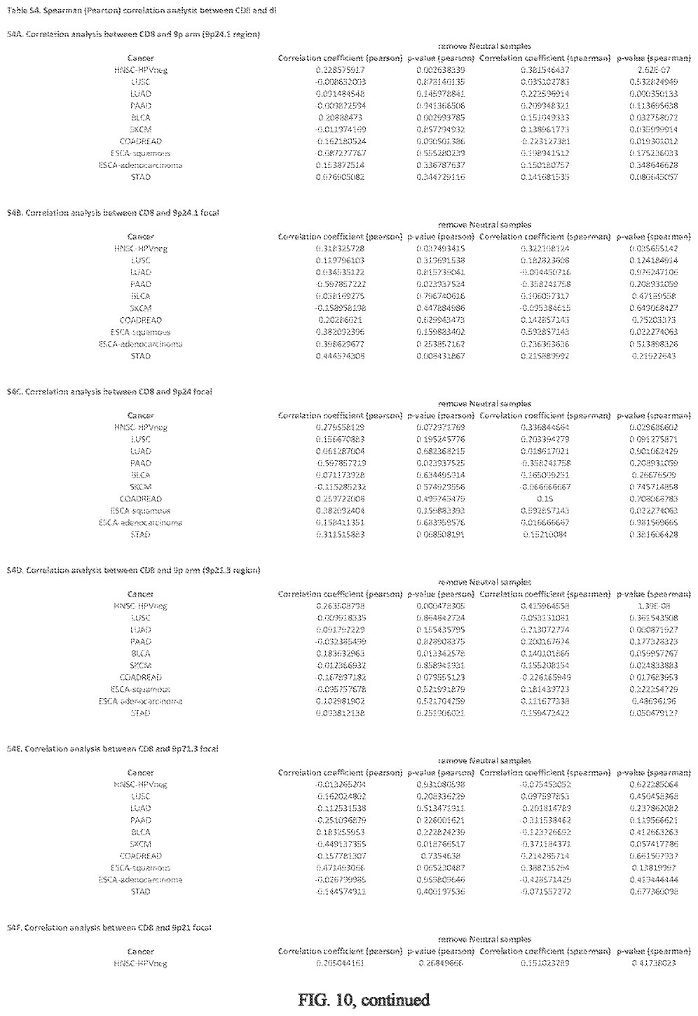

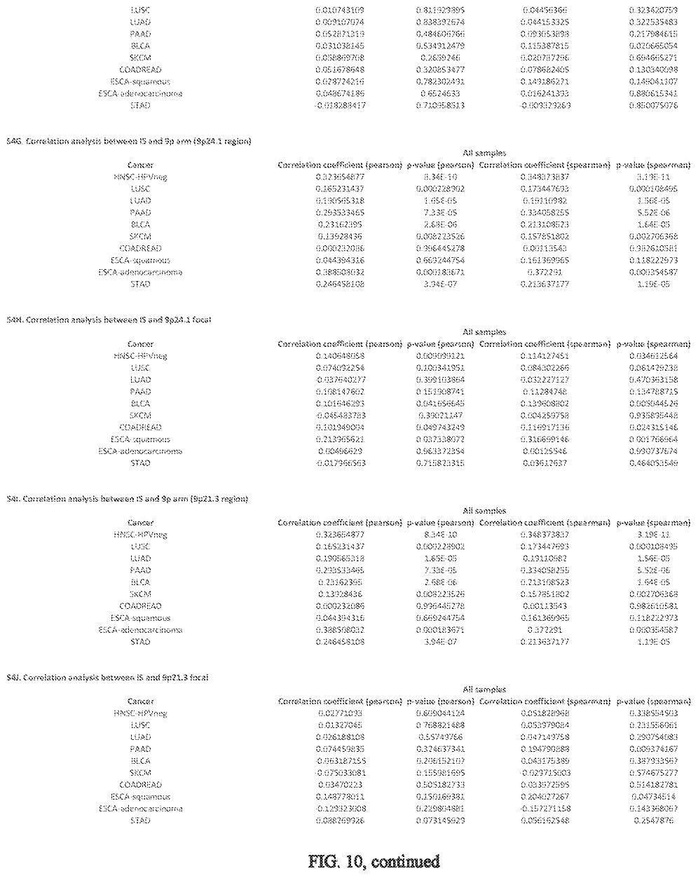

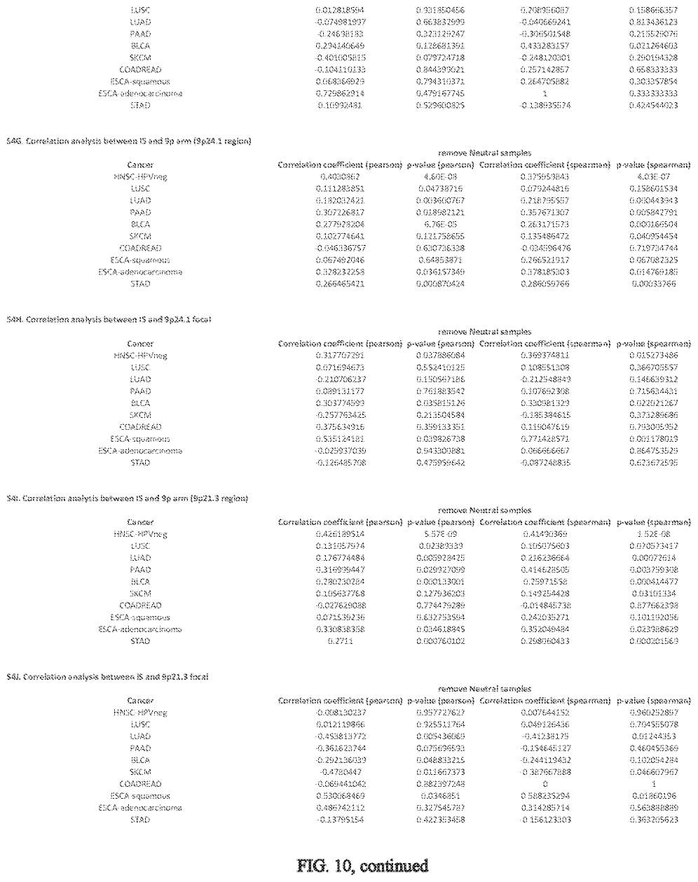

To better understand SCNA gene-dosage effects of 9p24.1 and 9p21.3, Applicant examined the correlation between CD8 T-cell and SCNA levels as a continuous variable (thus including both deep losses and high gains) for these two key bands. Applicant calculated these correlations both including all samples and after excluding the samples with no gain or loss (see Methods). 9p and 9p24.1 SCNAs showed a positive correlation with CD8 T-cell level (Spearman's rho-0.38, p=2.6E−7; and rho-0.32, p=0.04, respectively) after removal of samples with no gain or loss (FIG. 10, FIG. 1C). Similar results were found when Applicant tested the full 9p24 locus (which includes the three—9p24.1, 9p24.2 and 9p24.3—bands), where 9p24 SCNA showed a positive correlation with CD8 T-cell level (Spearman's rho=0.34, p=0.03 after removal of samples with no gain or loss). In contrast, none of the correlations between the 9p21 locus or 9p21 bands (9p21.1, 9p21.2, 9p21.3) and CD8 T-cell level showed statistical significance (Spearman's rho=−0.08, p=0.62 for 9p21.3; and rho=0.15, p=0.42 for 9p21). Accordingly, Applicant also found a positive correlation between CD8 T-cell level and JAK2-CD274 (located on 9p24.1) SCNA (Spearman's rho=0.40, p=2.0E−3, FIG. 1C), but not for CDKN2A-MTAP SCNA (located on 9p21.3), Spearman's rho=−0.15, p=0.08 for CDKN2A-MTAP (data not shown).

9p24.1 SCNA Associations with TME Phenotype in Independent HPV− HNSC Validation Cohort.

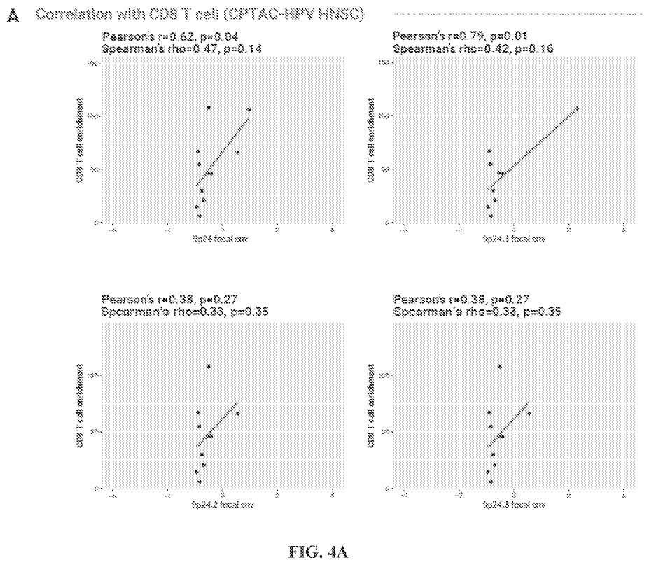

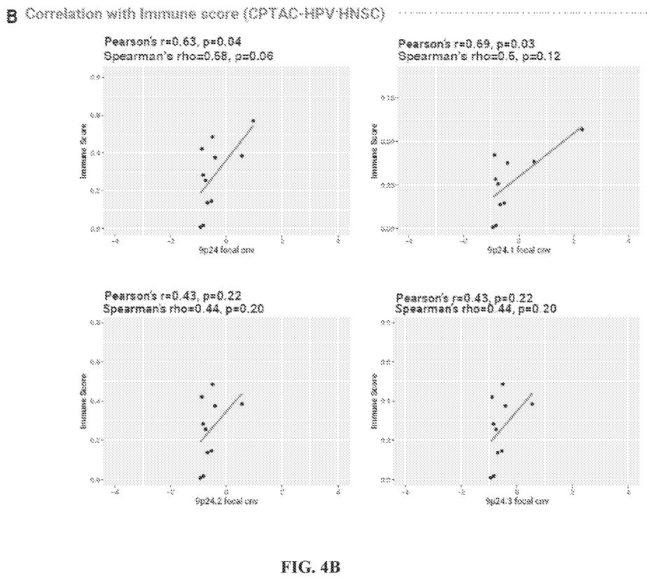

To validate Applicant's findings in an independent patient cohort, Applicant performed similar analyses on the HPV− HNSC cohort from CPTAC (Clinical Proteomic Tumor Analysis Consortium) (32). After adjusting the SCNA by purity and ploidy as in TCGA, 108 HPV− HNSC patients were available for the analysis. Among them, 20% had 9p loss and 8% had 9p24.1 focal loss. Applicant examined the correlation between CD8 T-cell level (and immune score) and SCNAs for 9p24 and 9p24.1 considered as continuous variables (including both losses and gains). There was a positive 9p24 trend for the correlation with CD8 T-cell level and immune score (Spearman's rho-0.58, p=0.06 for immune score). Similar positive trends were observed for 9p24.1, but not for 9p24.2 and 9p24.3 (FIG. 4), albeit limited by the small sample size of this dataset (N=10). Taken together, the results showed that an important SCNA contributor to the association between 9p and TME (CD8 T-cell level and immune score) was 9p24.1; Applicant's results attribute a less significant effect of 9p21.3 to TME (Spearman's rho=0.44, p=0.16 for CD8 T-cell level, data not shown).

### Tissue-Specific 9p21.3 and 9p24.1 Dosage Effects on TME Phenotype in Different Solid Cancers.

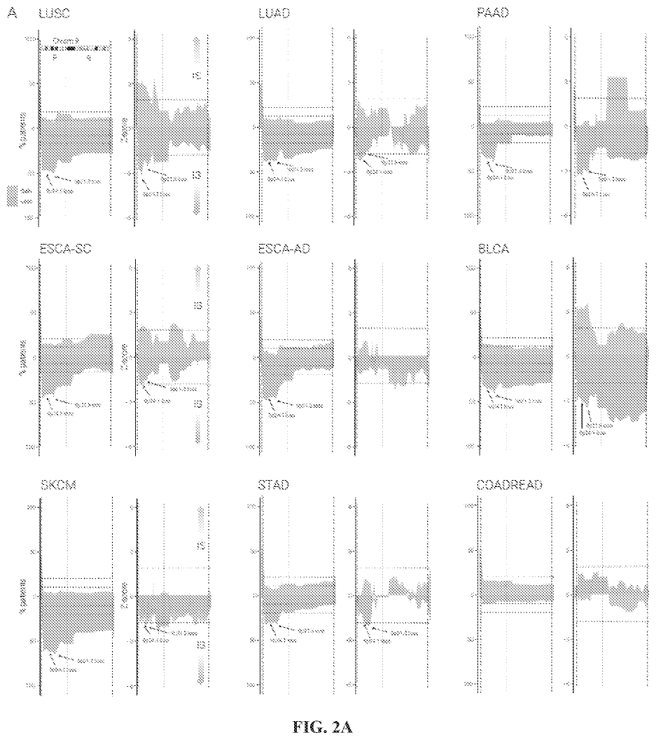

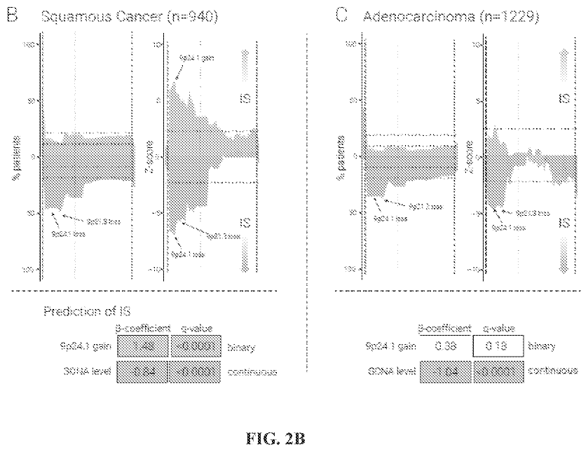

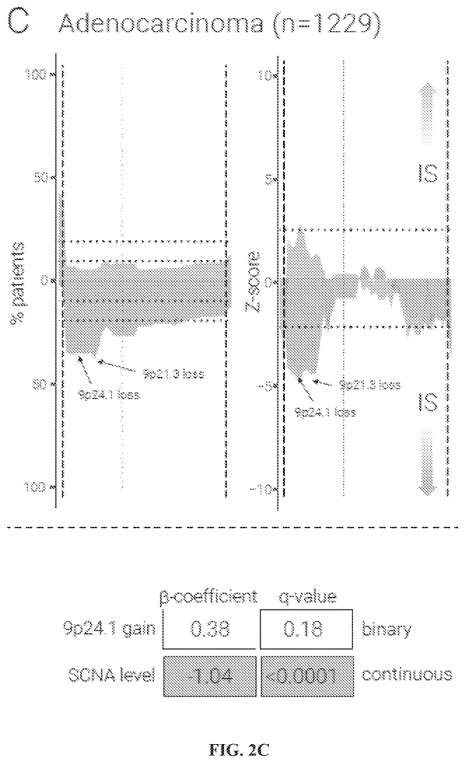

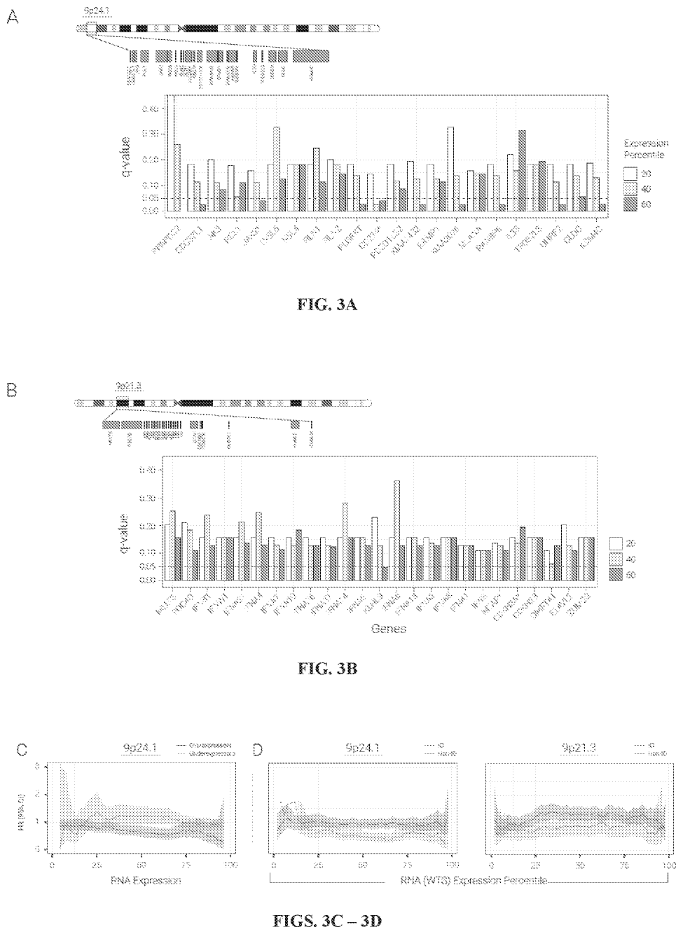

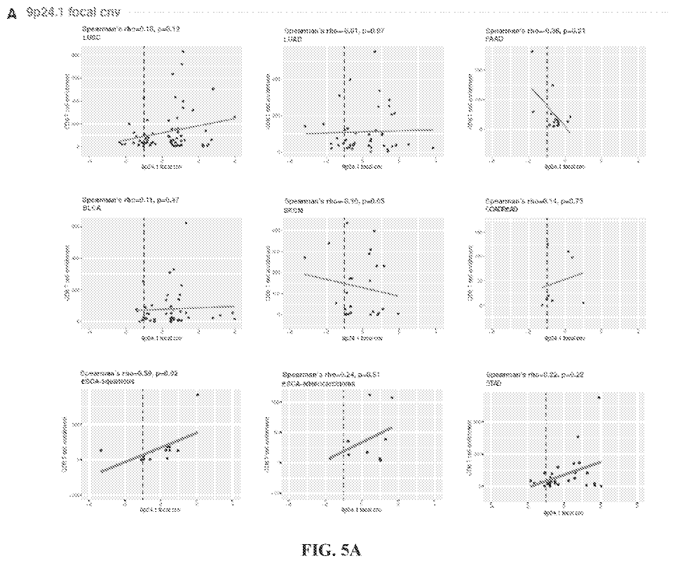

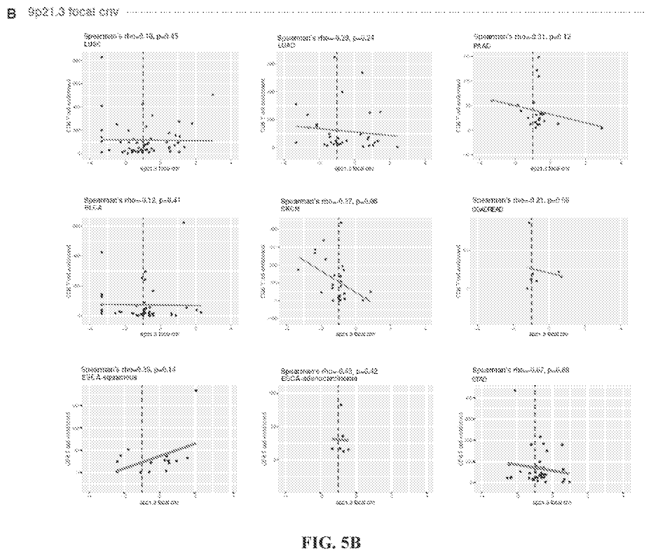

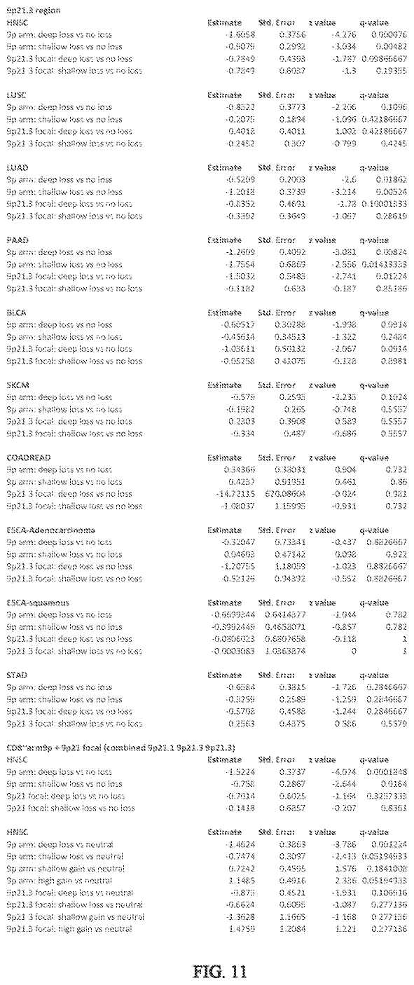

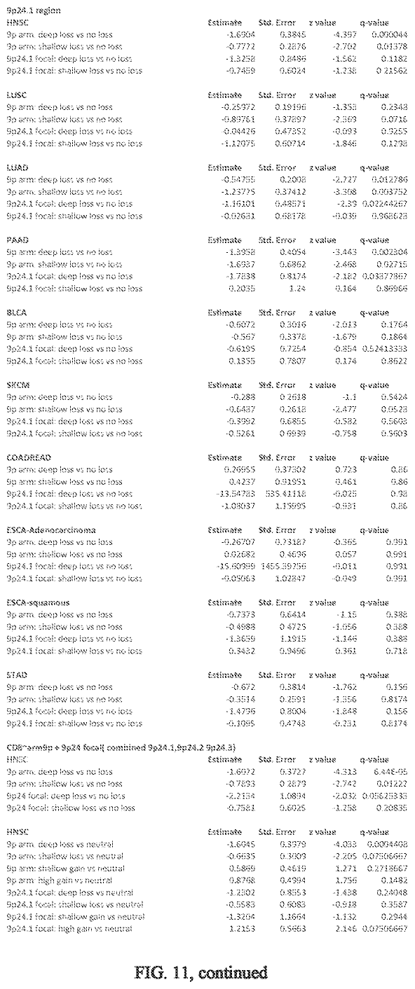

To examine 9p21.3 and 9p24.1 SCNA-frequency and immune-marker patterns across different solid tumor types, Applicant performed analyses similar to those above for HPV− HNSC on data derived from TCGA for nine other cancer types, eight selected with 9p-loss frequencies >25%, ranging from 29% for STAD (stomach adenocarcinoma) to 60% for SKCM (skin cutaneous melanoma) shown in Dataset S1. Applicant also included COADREAD (colorectal adenocarcinoma) as a control example of a common solid tumor type with infrequent (10%) 9p loss. Using similar methods to above (in FIGS. 1B-1D), Applicant found that in the eight tumors with frequent 9p-loss, this loss event was statistically significantly associated with lower cytotoxic immune score and CD8 T-cell levels (FIG. 2A, FIG. 7). When Applicant examined the two 9p bands individually, Applicant found that 9p24.1 loss was associated with lower immune scores in LUSC (lung squamous cell carcinoma), LUAD (lung adenocarcinoma), PAAD (pancreatic adenocarcinoma), BCLA (bladder cancer), ESCA (esophageal cancer)-Squamous, but not in ESCA-Adenocarcinoma, SKCM, STAD, or COADREAD (Dataset S1). In contrast, 9p21.3 loss was associated with lower immune scores in LUSC, PAAD, BLCA and STAD only. 9p24.1 gain, however, was associated with higher immune scores in LUSC, BLCA, and ESCA-squamous (FIG. 7). Taken together, these results show tissue specificity for SCNA/TME associations, with a broad association of overall SCNA level and 9p loss with immune-cold phenotypes in multiple cancers, a more prominent immune-cold effect of 9p21.3 loss in PAAD, and an association of 9p24.1 gain with immune-hot phenotypes restricted to squamous tumors including BLCA (33), most notable for LUSC, which was recently shown to cluster closely in SCNA profiles with HPV− HNSC (34). Indeed, the 9p24.1 gain/immune hot association was readily apparent when Applicant grouped squamous-cell cancer histologies (see arrows in FIG. 2B), but not evident in an analysis of adenocarcinomas combined (FIG. 2C; FIG. 5, FIGS. 7-11).

Whole Transcriptome Sequencing (WTS) Reveals 9p-Dosage, TME Correlates in HPV− HNSC.

Applicant evaluated the correlation of RNA expression from WTS to DNA copy number from WES across 9p band and gene levels in an independent HPV− HNSC cohort of 1746 patients. 9p24.1 gene dosage derived from WTS tracked closely with copy number determined by WES, with Spearman's rho coefficient of 0.746 (p<1.0E−4). Next, Applicant focused Applicant's WTS analyses on computing the associations of 9p24.1 or 9p21.3 with CD8 T-cell levels, and found that the 9p24. 1 transcript correlate, JAK2-CD274, was more highly correlated with CD8A/B levels (rho=0.61/0.55, p<1.0E−4) than the 9p21.3 correlate, MTAPCDKN2A (as recently reported by (3) for CD8A/B (rho=0.21/0.17). These results are consistent with the TCGA findings (FIG. 1C) supporting the hypothesis that 9p24.1 plays a larger role in HPV− HNSC TME activation than 9p21.3.

### 9p24.1 Transcriptomic Variation Predicts Anti-PD-1 ICT Benefit in HPV− HNSC.

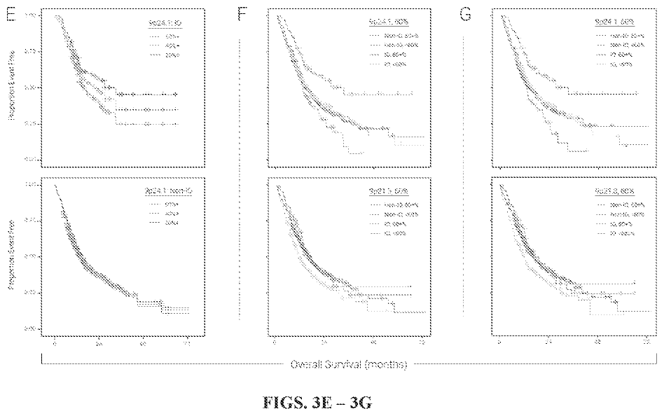

Based on the strong, consistent (from three cohorts above) 9p24.1 association with TME activation in WES and WTS datasets, Applicant hypothesized that 9p24.1 transcript level could represent a novel biomarker that, in addition to (and potentially in lieu of) PD-L1 protein expression, could more accurately predict clinical benefit from PD-1-targeted agents in HPV− HNSC. To test this hypothesis, Applicant analyzed 9p24.1 gene-dosage associations from WTS profiles with patient survival after immunotherapy in a deidentified, real world cohort (RWC) dataset of 894 HPV− HNSC patients with recurrent/metastatic disease (see Methods): 208 patients received first- or second-line anti-PD-1 checkpoint therapy (pembrolizumab, nivolumab), and 694 patients had been treated with chemotherapy (with no prior or subsequent immunotherapy). In Applicant's initial approach, focused on the ICT-treated group only, Applicant evaluated every gene in the 9p21.3 and 9p24.1 bands individually to determine if expression levels of each gene singly could stratify patient survival after ICT. Accounting for false discovery, of the 25 genes at 9p21.3, only one gene, at one percentile threshold (KLHL9 at the 60th percentile threshold) was significantly associated with survival (q-value<0.05), whereas 9 of the 22 genes at 9p24.1 were statistically significantly associated with ICT survival at the 60th percentile (Fisher's exact, p=0.005) (FIG. 3A and FIG. 3B). Importantly, many more 9p24.1 gene percentiles had statistically significant p-values that did not survive false discovery correction (data not shown); Applicant did not see similar survival patterns in 9p21.3. These data show an inherent 9p band-level difference in shaping immune response, and suggest that 9p24.1 is a relative hotbed of immune-regulatory genes.

Applicant next investigated the contributions of JAK2 or CD274 when analyzed individually or combined. Given the high correlation and co-linearity of JAK2 and CD274, Applicant plotted hazard ratio (HR) metrics from a Cox proportional hazards model for survival after ICT versus chemotherapy according to percentile expression of each gene alone or combined. The maximum HR differences between over- and under-expressors occurred at percentile 66th for JAK2-CD274 (FIG. 3C; the peak thresholds for CD274 alone and JAK2 alone, were 44, 70, respectively, data not shown). Applicant then defined a null hypothesis that the HR separations between the expression levels of CD274 or JAK2 are the same across all percentiles. With a p-value of 3.0E−3, Applicant were able to reject the null hypothesis, indicating that not only was the JAK2-CD274 peak HR difference greater than those for CD274 and JAK2 expressions alone, but that the signature patterns by expression percentile were different, and that the combination of them together provides ICT predictive information missed by either one alone. At this optimal threshold, patients treated with chemotherapy had the corresponding HRs of 0.9, 1.1, and 1.0, respectively, showing that the survival difference is dependent on, and specific for, administration of ICT. These findings are consistent with Applicant's earlier targeted sequencing study where Applicant observed a JAK2-CD274 co-deletion association with ICT resistance that was much stronger than either gene deletion alone (2). A Fisher's exact test checked whether Applicant expect the same signal shift (ICT better than non-ICT in overexpressors; opposite in underexpressors) in the same locations along the X-axis. Kaplan-Meier survival analyses at the optimal points provided an independent assessment and independent p-value correction (data not shown). For each percentile, Applicant assessed whether the overexpressor cohort showed the same survival benefit to ICT or standard therapy. When p<0.05, Applicant rejected the null hypothesis that the survival benefit is the same for the ICT and non-ICT treated cohorts. Among JAK2-CD274 over expressors at the 66^{th} percentile, the comparisons were significant at p<0.0005 and q=0.019, an FDR adjustment of a log-rank p-value (see SI Methods). At a standard 5% alpha on the FDR, Applicant expect that 5% of the evaluated percentiles that are called “significant” would actually be null (i.e., no survival difference). The observed q<0.05 indicates that the CD274+JAK2 signature at the 66th percentile is associated with a survival benefit in ICT-treated (relative to non-ICT) treated patients (FIG. 3C). ICT-treated patients with expression less than the defined cutoff had decreased survival compared to those treated with chemotherapy, although only CD274 less than the 44^{th} percentile achieved statistical significance (data not shown).

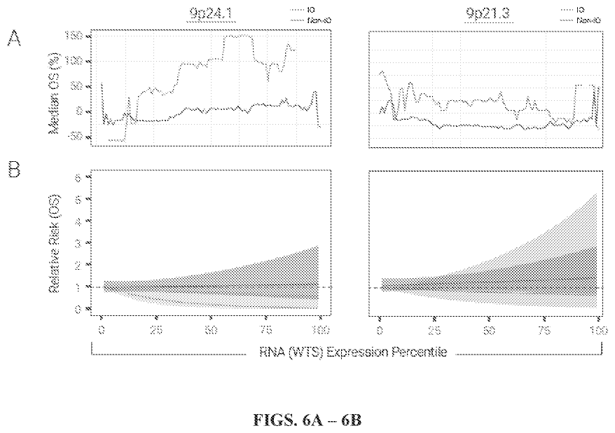

Applicant next computed HRs for survival as a function of 9p21.3 represented by the CDKN2A-MTAP transcript, as assessed in recent reports (3, 21) and for 9p24.1 (represented by JAK2-CD274) dosage percentiles in a continuous-variable analysis, using the lowest percentile as the reference group (FIG. 3D). Applicant found decreasing HRs with increasing JAK2-CD274 transcript expression dosage in ICT-treated patients (with the curves crossing HR=1 at 20^{th} expression percentile), but not in chemotherapy-treated patients. HRs remained relatively unchanged with increasing CDKN2A-MTAP expression dosage. Analyses of median overall survivals or relative risks of death showed similar patterns (FIG. 6A and FIG. 6B). These results support the role of 9p24.1 (but not 9p21.3) transcript downregulation or upregulation (and specifically JAK2-CD274) as a predictive biomarker of ICT resistance or sensitivity, respectively, in HPV− HNSC. Kaplan-Meier survival plots for the 20^{th}, 40^{th} and 60^{th} RNA percentiles for 9p24.1 treated with anti-PD-1 therapy (FIG. 3E) revealed superior survival of the top (vs bottom) 40% expression subgroup (HR=0.58, 95% CI: 0.387-0.873; log rank p-value=0.008, FIG. 3F); there were no significant differences by 9p24.1 expression percentile in the non-ICT (IO) group (HR=1.115, CI: 0.899-1.383, FIG. 3F). The selective predictive effects of JAK2-CD274 transcript expression is shown by the inferior Kaplan-Meier survival curve of ICT-treated patients (compared to chemotherapy-treated patients) in the <60^{th} percentile subgroup, in sharp contrast to the superior survival of ICT-monotherapy in the subgroup with the highest 40^{th} transcriptome dosage percentile (FIG. 3F). Similar results were observed when using the 3-gene amplicon (including PD-L2) at 9p24.1 (data not shown). Notwithstanding the prominent roles observed with genes at 9p24.1, there is an apparent influence of larger deletions and 9p21.3 gene-level contribution as well, as KLHL9 from 9p21.3 was statistically significant at the 60% threshold after false discovery correction (FIG. 3B). KLHL9 expression added to the median overall survival difference observed for JAK2-CD274 (data not shown). Because PD-L1 IHC protein expression is routinely used in clinical practice to select patients for ICT, Applicant assessed whether JAK2-CD274 transcriptome dosage could further identify PD-L1-positive patients most likely to benefit from ICT or chemotherapy. Within the 803 (of the total 894 RWC) subgroup of patients with standard binary PD-L1 combined positive score (CPS) protein expression≥1, JAK2-CD274 transcript levels <60 percentile identified PD-L1 IHC-positive patients with survival rates inferior to chemotherapy (FIG. 3G).

### Discussion

HPV− head and neck cancer, the most common and lethal subtype of head and neck cancer with over 200,000 deaths globally per year, is characterized by extensive somatic genomic copy-number alterations. Here, Applicant demonstrated that 9p24.1-genetic dosage significantly contributed to an immunecold or hot phenotype (when genes are lost or gained, respectively) in HPV− HNSC, in WES and WTS analyses of three independent cohorts, which in turn predicted resistance and sensitivity to standard anti-PD-1 ICT in a fourth real-world patient cohort with recurrent/metastatic disease. The contributions of 9p21.3 to immune TME activation and ICT response were less prominent or non-existent. These data build on Applicant's previous report demonstrating that 9p somatic copy-number loss in HPV− HNSC was associated with immune-cold tumor microenvironments and poor survival after anti-PD-1 immunotherapy (2). There have been two subsequent solid-tumor studies of 9p21.3 loss (inferred from two genes on this band, CDKN2A and MTAP) reporting that 9p21.3 loss was associated with TME and/or ICT outcomes in lung adenocarcinoma, bladder cancer, melanoma and small mixed solid-tumor ICT cohorts (3, 21). Both reports included too few patients (17 in each report, HPV-status unclear) with HNSC to analyze separately but these HNSC patients were included in ICT outcome analysis of mixed solid-tumor cohorts. The largest study was a pan-tumor study of CDKN2A and MTAP expression as a surrogate for 9p21.3 heterozygous or homozygous deletion, which confirmed Applicant's earlier HNSC/TME findings (3), specifically showing that 9p21.3 loss in HPV− HNSC was associated with immune-cold, CD8 T-cell depleted TME. This latter report showed no difference between heterozygous LOH and homozygous 9p21.3 deletion on TME in HPV− HNSC (and most other solid tumors studied), and the authors speculated that 9p24.1 may be co-lost with 9p21.3. The second smaller study of 9p21.3 ‘deep’ deletions (21) reported inferior survival trends in an analysis of mixed solid-tumor cohort of 87 total patients. A third recent study reported that 9p21.3 loss (as assessed by CDKN2A, CDKN2B plus MTAP) was associated with poor survival after anti-PD-1 monotherapy, but not in ICT-chemotherapy combination treated patients with non-squamous lung cancer (35). The ICT-monotherapy findings remained, albeit less statistically significant, in a subgroup of PD-L1-positive patients.

Previous work from Applicant's group and others have demonstrated that 9p21.3 and 9p loss are among the most frequent focal and arm events in human cancer (19, 20). In the current report, Applicant assessed somatic 9p-band alterations as a continuous variable, and demonstrated that 9p24.1 loss was associated with immune-cold, CD8 T-cell depleted HPV− HNSC, whereas 9p21.3 focal loss was not. Both 9p21.3 and 9p24.1 loss and gain frequencies were similar, and frequently occurred as part of an arm-level event which could confound previous analyses on the specific influence of regional 9p21.3 alterations on immune TME activation when such effects were indeed due to co-alterations in 9p24.1. Applicant's analyses (1) support the hypothesis that 9p24.1 is a somatic alteration key to shaping the immune TME response, likely with more modest contributions of alterations in genes located elsewhere in 9p (e.g., KLHL9, FIG. 3B) and other chromosomes, (2) justify the development of 9p-related biomarker tests, more specifically, 9p24.1 (or JAK2-CD274 transcriptomic correlates), as more efficient biomarker tests to select patients for ICT. Importantly, not only did Applicant confirm and extend binary 9p loss/immune-cold/ICT resistance observations in Applicant's and other recent reports, Applicant demonstrated 9p24.1 gain as a possible driver of an immune activation and ICT response in squamous cancers.

The mechanisms behind somatic 9p24.1 dosage effects on immune TME remain to be elucidated. PD-L1 expression is often considered a result of a downstream effect from INF-γ signaling in the context of immune infiltration—PD-L1 loss or gain alone would, therefore, be unlikely to directly influence TME, even though it could determine how tumors escape following immune activation. Specific 9p24.1 alterations relevant to both TME and response/resistance to immunotherapy include the IFN-γ pathway gene JAK2. JAK2 gain or loss of function somatic alterations promote or suppress PD-L1 expression, respectively, which affect TME and ICT response. In contrast to PD-L1, one could postulate a direct, broad effect of JAK2 alterations on TME, and pivotal role of JAK2 in cancer-cell sensitivity to IFN-γ, impaired T-cell sensitivity, and evasion (36), by modulating the degree of PD-L1 expression and antigen presentation upon IFN-γ release (37) further augmenting or dampening immune response. As an example, in triple negative breast cancer cell lines with 9p24.1 gain, PD-L1 expression was markedly inducible by low-dose IFN-γ in a copy-number dependent manner, mimicking an in situ inflammatory response (38). An enhanced, PD-L1 enriched, inflammatory response could explain the immune-hot phenotype observed in HPV− HNSC with 9p24.1 gain in Applicant's study, consistent with a recent report of the CD8+ T-cell inflamed phenotype in HNSC samples enriched by CD274, PDCD1KG2, JAK2 and KDM4C at 9p24.1 amplification (39). The 9p24.1 gain/immune-hot association seems to be tissue specific and prominently featured in squamous-cell histologies, primarily driven by HPV− HNSC and squamous-cell carcinomas of the lung (FIG. 2), which has been reported to track experimentally and computationally with HPV− HNSC in pan-cancer genomic-SCNA association studies (33, 34), possibly reflecting shared coevolution of immune evasion and neoplastic invasion (1, 25, 40-42). This highlights the importance of determining mechanisms of 9p band somatic alteration-related immune modulation in different tumor types, especially if these genomic features are to be used as biomarker tests to guide precision ICT. In pancreatic cancer, for example, Applicant found that 9p21.3 loss was the prominent driver of low IS/CD8 T cells, in accordance with recent evidence in pancreatic cancer mouse models (43).

The strong associations between 9p24.1 gene dosage and immune TME open the opportunity for biomarker development to guide ICT in HPV− HNSC and other tumor types. In support of this application, Applicant demonstrated that high expression levels of nearly half of the genes in 9p24.1 were associated with ICT benefit, whereas only one gene in 9p21.3 was (FIGS. 3A and 3B), prompting reexamination of the role of 9p21.3 as a predictive marker in HPV− HNSC. Two recent studies correlated CDKN2A/MTAP loss as an ICT resistance marker in several pan cancers but were limited by the small number of HPV− HNSC patients (3, 21) and could not evaluate the contributions and interactions of other chromosomal sites/genes to this observation. Applicant's large RWC dataset analyses, pointed to the strong survival associations of JAK2-CD274 dosage, with anti-PD-1 monotherapy producing a threefold increase in median survival at the 60th-percentile expression, and above, threshold.

Although Applicant found consistent, statistically significant associations of CD274 and JAK2 with anti-PD-1 response in Applicant's HPV− HNSC RWC, Applicant's data strongly support the IO importance of several other genes at 9p24.1 including RANBP6 and KDM4C, amplification of the latter gene was associated with TME hot in a recent HNSC report (39), rather than CDKN2A-MTAP, limited to ICT (not chemotherapy) treated patients. Notably, the highly selective predictive effects of JAK2-CD274 transcript levels were bidirectional, and could identify patients that had ICT outcomes inferior to chemotherapy in the low expression groups, in resonance with the somatic alterations immune cold/hot phenotype associations.

These 9p24.1 effects were evident even within the group with PD-L1 CPS ≥1 (FIG. 3G), suggesting this to be a novel biomarker test to refine the predictive value of PD-L1 alone to assess resistance and sensitivity to ICT in HPV− HNSC. Finally, since the 9p arm-level loss is the strongest predictor of TME in HPV− HNSC (Dataset S1) as well as other tumor types, there are likely other genes on 9p that cooperate with genes on 9p24.1 to promote immune cold TME.

In summary, this report provides multi-omic evidence from four cohorts that the spectrum of immune TME alterations from cold to hot in HPV− HNSC are highly influenced by somatic alterations in 9p24.1 dosage in HPV− HNSC, with 9p21.3 genes playing a secondary role. The wide application and low response rates of ICT makes it imperative to develop biology-driven, accurate biomarker tests that clinicians can use to help predict and guide therapy (or at least be complementary to other measures), thus sparing toxic, costly, and potentially non-efficacious treatment. When taking the complexity of the immune system into consideration, it is becoming increasingly evident that Applicant will have to integrate, through comprehensive multi-omic immune evaluations, both genomic and nongenomic biomarkers in the predictive tools for ICT response/resistance, if all aspects of the cancer-immunity cycle are to be encompassed in decision-making algorithms. Within this context, JAK2-CD274 expression may need to be incorporated, in addition to standard binary PD-L1 immunohistochemistry, into anti-PD-1 ICT-based strategies to maximize precision treatment, not only for HNSC, but potentially other (squamous) solid tumors in which 9p24.1 dosage shapes TME.

### Methods

### Datasets in TCGA.

Arm and gene level somatic copy number alteration (SCNA), gene expression, HPV status, and clinical parameters for HPV− HNSC (Head and Neck cancer), LUAD (Lung adenocarcinoma), LUSC (Lung squamous cell carcinoma), PAAD (Pancreatic adenocarcinoma), BLCA (Bladder Urothelial Carcinoma), SKCM (Skin Cutaneous Melanoma), ESCA-Squamous (Esophageal carcinoma), ESCA-Adenocarcinoma (Esophageal carcinoma), STAD (Stomach adenocarcinoma) and COADREAD (Colorectal adenocarcinoma) were derived from TCGA dataset. Copy number (given as log 2 copy number ratios) data for the 9 cancers above in TCGA cohort were derived from Affymetrix SNP 6.0 arrays and obtained from GISTIC2 analysis (Level 4). Gene expression file were obtained from RSEM analysis (Level3). All TCGA related files can be downloaded from Broad GDAC Firehose (v20160128). See SI Appendix for additional Methods details, including SCNA classification, variable importance analysis, CD8 T-cell deconvolution, associations between CD8 T-cell and SCNA levels, and associations between immune score and SCNA levels.

### Datasets in CPTAC.

For CPTAC3-HNSC (32), log 2 ratio gene level and segment SCNA as well as gene expression and clinical parameters used in the analysis can be obtained from LinkedOmics (http://www.linkedomics.org). The HPV status was filtered by clinical parameters “HPV inference.” GISTIC2 (44) was applied to generate the 9p arm and 9p24.1, 9p24.2, 9p24.3 focal level SCNA with default parameters. 9p24 and 9p21 SCNA was evaluated based on the median of genes located on 9p24.1, 9p24.2 and 9p24.3; and 9p21.1, 9p21.2 and 9p21.3, respectively.

Dataset from Real World Cohort (RWC).

A total of 1746 HPV− HNSC cases with whole transcriptome sequencing (WTS) and whole exome sequencing (WES) data, and a total of 894 HPV− HNSC cases with WTS and outcomes data available were used for this study. Immune hot was defined as WTS TPM for CD8A and CD8B both being greater than the median TPM values for CD8A and CD8B, respectively. See SI Appendix and Datasets for additional analyses including DNA copy number estimation, correlation analysis between WTS and WES, and Real-World Cohort analysis.

### Experiment No. 2

### SCNA Classification in TCGA and CPTAC.

The copy number for each chromosomal region (given as log 2 copy number ratios) were adjusted by tumor purity and ploidy derived from previously reported ABSOLUTE (1) methods. Arm-level copy-number related purity and ploidy was downloaded from TCGA Firehose Legacy (https://gdac.broadinstitute.org). For CPTAC, purity was determined from column “tumor pathology review” and ploidy is generated based on the mean ploidy of HPV− HNSC in TCGA. Then for each patient, purity a, ploidy t, integer copy number q (x) together with arm-level copy number R (x) was applied to the SCNA adjustment:

$${R}^{′} ( x ) = \frac{q ( x )}{τ} = \frac{α * τ * R ( x ) + 2 ( 1 - α ) * R ( x ) - 2 ( 1 - α )}{α * τ}$$

After purity adjustment, Applicant consider a log 2-transformed copy-number ratio >0.3 as a gain event and < (−0.3) as a loss event. To distinguish between arm and focal-level events, Applicant considered a threshold of ≥70% (default value in GISTIC2) of arm length (given in units of the fraction of chromosome arm) to identify the arm-level events (as defined in Davoli et al., 2017 (2)) while all the others were considered as focal-level events. The arm level, arm or focal level and focal level data were generated based on GISTIC2 output. In a more detailed way, the arm level SCNA was derived from “broad_values_by_arm.txt”. The arm or focal level SCNA was evaluated by the median of genes on the same region from file “all_data_by_genes.txt”. For example, the SCNA for region 9p21.3 is based on the median SCNA of 25 genes located on 9p21.3. The SCNA for region 9p21 is based on the median of genes located on 9p21.1, 9p21.2 and 9p21.3. The focal region level SCNA was calculated by the median of genes on the same region from file “focal_data_by_genes.txt”.

For JAK2-CD274 SCNA in HPV− HNSC, Applicant first calculated the mean SCNA of JAK2 and CD274 for each patient, then Applicant determined the gain and loss of JAK2-CD274 at 9p24.1 based on a cutoff of 0.3 (>0.3 as gain and < (−0.3) as loss), after that Applicant calculated the Spearman correlations between JAK2-CD274 SCNA and CD8 T-cell level. The same analysis was done for CDKN2A-MTAP at 9p21.3.

While in certain studies, tumor SCNAs are defined as homozygous or heterozygous, this holds primarily for germline SCNA. Defining whether cancer cells contain heterozygous (i.e., loss of one copy) or homozygous (i.e., loss of two copies) deletion is difficult. While the genome of normal cells is generally diploid, in most of the cancer cells from solid tumors the karyotype is highly aneuploid. For example, based on ABSOLUTE estimates (1), Applicant found that HPV− head and neck cancer contains a mean of 2.6 copies per chromosome, which means each chromosome is represented by almost three copies, or triploid, instead of two as in normal cells. Thus, for cancer, instead of heterozygous and homozygous loss, Applicant considered ‘shallow’ and ‘deep’ loss. More specifically, Applicant consider tumors with log 2 copy number ratio less than −1 as a deep loss (meaning that at least 50% of the chromosome copy number is lost), corresponding to 1 copy lost for a diploid genome, 1.5 copies for a triploid genome or 2 copies for a tetraploid genome (FIG. 7). On the other hand, Applicant considered log 2 copy-number ratio between −1 to −0.3 as shallow loss (meaning that at least 20% of the chromosome copy number is lost), corresponding to 0.4 copy loss for a diploid genome, 0.6 copy for a triploid genome or 0.8 copy loss for a tetraploid genome). Applicant also analyzed the tumors using the ‘homozygous’ and ‘heterozygous’ definitions reported earlier, and the results were similar to Applicant's findings using the ‘deep’ and ‘shallow’ loss definitions above. Furthermore, Applicant also determined tumors with log 2 ratio more than 0.65 as high gain (meaning that at least 50% of the chromosome copy number is gained), 1 copy gain for diploid genome, 1.5 copies for triploid genome and 2 copies gain for a tetraploid genome; and log 2 copy number ratio between 0.3 to 0.65 as shallow gain (meaning 25% of the chromosome is gained), corresponding to 0.5 copy gain for a diploid genome, 0.75 copy for a triploid genome or 1 copy gain for a tetraploid genome. Copy number neutral was classified as log 2 ratio between −0.3 to 0.3.

The total SCNA level were defined as numbers of arms with gains or losses (see formula below):

$$SCNA level = \sum arm gains + \sum arm losses$$

### Association Between Immune Score and SCNA in TCGA and CPTAC.

The immune score (IS) was generated from cytotoxic immune cells markers: GZMH, PRF1, CD3E, CD247, CD2, GZMK and NKG7 (2). In brief, for each marker Applicant ranked the gene expression across all patients belonging to same cancer and Applicant summed the rank order to get a new list and then ranked the new list to generate the immune score. Next, Applicant defined the tumors as having a low or high immune score using the bottom 35th and top 65th percentiles. For each SCNA, Applicant used independent evaluation to analyze the association between immune score and gain or loss separately. The normalized SCNA level was also applied to the model. Then Applicant examined the association between immune score and SCNA by applying a multivariable logistic model for gains and losses separately (below). Applicant used z-scores to evaluate the association between immune score and SCNA. Positive and negative z-scores indicate that the SCNA is positively or negatively associated with the immune score; all the p-values from the model were adjusted by false discovery rate (3).

$$\begin{matrix}IS \sim SCNA ( gain ) + SCNA level \\ IS \sim SCNA ( loss ) + SCNA level \\ IS \sim 9 p 21.3 arm or focal loss level + 9 p 24.1 arm or focal loss\end{matrix}$$

### Variable Importance Analysis in TCGA.

In order to understand which covariant plays a more important role in regressions, Applicant used the varImp function from the caret package (v6.0-90) to calculate the overall importance, then Applicant used the weighted overall importance to calculate the final size-effect.

### CD8 T-Cell Deconvolution in TCGA and CPTAC.

Applicant used 6 different methods or markers to evaluate the CD8 T-cell enrichment. 1) The R package MCP-counter (4), which is based on the normalized log 2-transformed expression matrix to infer the absolute abundance scores for 8 immune cells (including CD8 T cells level). 2) quanTIseq (5), a method applied to the normalized RNA-seq data to estimate the relative proportions of 10 different immune cells (including CD8 T cells level). 3) CIBERSORT, a method applied to the normalized RNA-seq data to estimate the relative proportions of 22 immune cell subpopulations (including CD8 T cells level) using compartment-specific gene expression signatures (6). 4) xCELL, a webtool to perform cell type enrichment analysis from RSEM gene expression data for 64 immune types (including CD8 T cells level) (7). 5) RNA expression levels for CD8A. 6) RNA expression for CD8B; previous studies have shown that RNA expression levels (CD8A or CD8B) of immune-cell markers highly correlate with CD8+ T-cell estimates based on immunofluorescence (8).

### Associations Between CD8 T-Cell and SCNA Levels in TCGA and CPTAC.

The different CD8 T-cell enrichment associations with deep loss, shallow loss and no loss for 9p, 9p21 or 9p24 was calculated using the Wilcoxon rank-sum test and adjusted by the false discovery rate. In addition, Applicant defined the tumors as having low or high CD8 T-cell levels using the bottom 35th and top 65th percentiles. For each SCNA, Applicant used independent evaluations to analyze the associations between CD8 T-cell levels and gain or loss separately. Applicant used z-scores to evaluate the association between CD8 T-cell and SCNA levels. A positive z-score indicates that the SCNA is positively associated with CD8 T-cell levels, a negative z-score indicates that the SCNA is negatively associated with CD8 T-cell levels. All the p-values from the model were also adjusted by false discovery rate:

$$\begin{matrix}CD 8 T cell \sim SCNA ( gain ) + SCNA level \\ CD 8 T cell \sim SCNA ( loss ) + SCNA level \\ CD 8 T cell enrichment \sim 9 p arm loss + 9 p 21 \text{.3} focal loss \\ CD 8 T cell enrichment \sim 9 p arm loss + 9 p 24 \text{.1} focal loss \\ CD 8 T cell enrichment \sim 9 p 21.3 arm or focal loss level + 9 p 24.1 arm or focal loss\end{matrix}$$

### Statistical Analysis for TCGA and CPTAC.

In addition to the bioinformatics approaches described above, a Spearman's or Pearson's correlation analysis was used to identify CD8 T-cell enrichment correlations with 9p, 9p21.3 or -9p24.1 SCNAs by using continuous values. All data were analyzed in R (v4.1.1).

### WES Pipeline for RWE Cohort.

Genomic tumor DNA isolated from formalin-fixed paraffin-embedded (FFPE) tumor samples was micro-dissected to enrich tumor purity and subjected to NGS using the NovaSeq 6500 platform (Illumina, Inc., San Diego, CA). FFPE specimens underwent pathology review to measure percent tumor content and tumor size; a minimum of 20% of tumor content in the area for microdissection was required to enable enrichment and extraction of tumor-specific DNA. Matched normal tissue was not sequenced. A custom-designed SureSelect XT assay was used to enrich whole exome whole-gene targets (Agilent Technologies, Santa Clara, CA). Somatic copy number alteration (SCNA) was tested by NGS and was determined by using CNVkit (9).

### WTS Pipeline for RWE Cohort.

FFPE specimens underwent pathology review to measure percent tumor content and tumor size; a minimum of 20% of tumor content in the area for microdissection was required to enable enrichment and extraction of tumor-specific RNA. For transcription counting, transcripts per million molecules was generated using the Salmon expression pipeline (10). RNA expression, as defined by transcripts per million (TPM) from the Salmon RNA expression pipeline (10) using the-clinically validated Caris Whole Transcriptome Assay (11).

### Gene Level Transcriptomic Variation in 9p24.1 and 9p21.3.

For genes on region 9p24.1 and 9p21.3 that have both RNA expression and survival data: First, Applicant split the patients into 3 independent groups based on different expression percentiles (>20^{th} percentile vs<20th percentile; >40th percentile vs<40th percentile; >60th percentile vs<60th percentile). Then a Wilcoxon rank test was applied for each comparison. All the p-values from-the results were also adjusted by the false discovery rate.

### 9p24.1 Immune Hot (Cold) Copy Number Estimation for Different RNA Expression in RWE.

For RWE dataset: Immune hot is defined as WTS TPM for CD8A and CD8B both being greater than the median TPM values for CD8A and CD8B respectively. For the RNA expression, cases were partitioned into deciles based on the median TPM of 22 genes (AK3, CD274, CDC37L1, ERMP1, GLDC, IL33, INSL4, INSL6, JAK2, KDM4C, KIAA1432, KIAA2026, MLANA, PDCDILG2, PLGRKT, PPAPDC2, RANBP6, RCL1, RLN1, RLN2, TPD52L3, UHRF2) located on 9p24.1. Then, the mean CNVKit copies of 9p24.1 was calculated within each decile, a Wilcoxon test was applied to-test the SCNA difference between immune hot and immune cold.

### Real-World Cohort Survival Analysis.

Applicant extracted the overall survival (OS) of each case from a repository of real-world evidence (RWE), defined as the biopsy date through last contact. Patients that did not have an observed claim data element within 100 days of the end of Applicant's RWE records were presumed to be deceased and uncensored, which was found to be 95% concordant to data obtained from National Death Index data (National Center for Health Statistics, Centers for Disease Control and Prevention). All other patients were considered censored. Patients with an observed anti-PD-1 checkpoint therapy were annotated as IO-treated, and all other patients were grouped into non-IO treated. PD-L1 protein expression by immunohistochemistry was determined using the 22C3 PD-L1 antibody, and a combined positive score ≥1 was considered positive for survival analysis, as previously described (12).

Hazard ratios for survival (and 95% confidence intervals) were computed for each percentile gene expression (where indicated) using the Cox proportional hazards model. Overall survival between groups was compared using the log-rank test.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Tables 1 to 5 to be inserted

TABLE 1

XZ: 0000-0001-8502-0139
EEWC: 0000-0002-9872-6242
WNW: 0000-0001-5008-8854
JJB: 0000-0003-1268-9637
JA: 0000-0001-5038-1735
DM: 0000-0001-7352-7485
DBS: 0000-0002-9531-0970
JSG: 0000-0002-5150-4482
LBA: 0000-0003-3596-4515
WKC: 0000-0003-3804-9179
SML: 0000-0002-1643-4124
TD: 0000-0003-4116-9745

Other aspects are set forth within the following claims.

### REFERENCES FOR EXPERIMENT NO. 1

1. T. Davoli, H. Uno, E. C. Wooten, S. J. Elledge, Tumor aneuploidy correlates with markers of immune evasion and with reduced response to immunotherapy. Science 355 (2017).
2. W. N. William, Jr. et al., Immune evasion in HPV (−) head and neck precancer-cancer transition is driven by an aneuploid switch involving chromosome 9p loss. Proc Natl Acad Sci USA 118 (2021).
3. G. Han et al., 9p21 loss confers a cold tumor immune microenvironment and primary resistance to immune checkpoint therapy. Nat Commun 12, 5606 (2021).
4. E. E. W. Cohen et al., The Society for Immunotherapy of Cancer consensus statement on immunotherapy for the treatment of squamous cell carcinoma of the head and neck (HNSCC). J Immunother Cancer 7, 184 (2019).
5. E. E. W. Cohen et al., Pembrolizumab versus methotrexate, docetaxel, or cetuximab for recurrent or metastatic head-and-neck squamous cell carcinoma (KEYNOTE-040): a randomised, open-label, phase 3 study. Lancet 393, 156-167 (2019).
6. R. L. Ferris et al., Nivolumab for Recurrent Squamous-Cell Carcinoma of the Head and Neck. N Engl J Med 375, 1856-1867 (2016).
7. B. Burtness et al., Pembrolizumab alone or with chemotherapy versus cetuximab with chemotherapy for recurrent or metastatic squamous cell carcinoma of the head and neck (KEYNOTE-048): a randomised, open-label, phase 3 study. Lancet 394, 1915-1928 (2019).
8. K. Emancipator et al., Comparing programmed death ligand 1 scores for predicting pembrolizumab efficacy in head and neck cancer. Mod Pathol 34, 532-541 (2021).
9. B. Burtness et al., Pembrolizumab Alone or With Chemotherapy for Recurrent/Metastatic Head and Neck Squamous Cell Carcinoma in KEYNOTE-048: Subgroup Analysis by Programmed Death Ligand-1 Combined Positive Score. J Clin Oncol 40, 2321-2332 (2022).
10. M. Ayers et al., IFN-γ-related mRNA profile predicts clinical response to PD-1 blockade. J Clin Invest 127, 2930-2940 (2017).
11. L. B. Alexandrov et al., Signatures of mutational processes in human cancer. Nature 500, 415-421 (2013).
12. A. Marabelle et al., Association of tumour mutational burden with outcomes in patients with advanced solid tumours treated with pembrolizumab: prospective biomarker analysis of the multicohort, open-label, phase 2 KEYNOTE-158 study. Lancet Oncol 21, 1353-1365 (2020).
13. M. M. Boyiadzis et al., Significance and implications of FDA approval of pembrolizumab for biomarker-defined disease. J Immunother Cancer 6, 35 (2018).
14. D. T. Le et al., Mismatch repair deficiency predicts response of solid tumors to PD-1 blockade. Science 357, 409-413 (2017).
15. W. Liao et al., KRAS-IRF2 Axis Drives Immune Suppression and Immune Therapy Resistance in Colorectal Cancer. Cancer Cell 35, 559-572 e557 (2019).
16. W. Peng et al., Loss of PTEN Promotes Resistance to T Cell-Mediated Immunotherapy. Cancer Discov 6, 202-216 (2016).
17. S. Spranger, R. Bao, T. F. Gajewski, Melanoma-intrinsic beta-catenin signalling prevents antitumour immunity. Nature 523, 231-235 (2015).
18. J. M. Zaretsky et al., Mutations Associated with Acquired Resistance to PD-1 Blockade in Melanoma. The New England journal of medicine 375, 819-829 (2016).
19. R. Beroukhim et al., The landscape of somatic copy-number alteration across human cancers. Nature 463, 899-905 (2010).
20. T. Davoli et al., Cumulative haploinsufficiency and triplosensitivity drive aneuploidy patterns and shape the cancer genome. Cell 155, 948-962 (2013).
21. P. Spiliopoulou et al., All is not lost: learning from 9p21 loss in cancer. Trends Immunol 43, 379-390 (2022).
22. O. Alhalabi et al., MTAP deficiency creates an exploitable target for antifolate therapy in 9p21-loss cancers. Nat Commun 13, 1797 (2022).
23. P. S. Linsley, C. Speake, E. Whalen, D. Chaussabel, Copy number loss of the interferon gene cluster in melanomas is linked to reduced T cell infiltrate and poor patient prognosis. PLOS One 9, e109760 (2014).
24. J. Gao et al., Loss of IFN-γ Pathway Genes in Tumor Cells as a Mechanism of Resistance to Anti-CTLA-4 Therapy. Cell 167, 397-404.e399 (2016).
25. W. Roh et al., Integrated molecular analysis of tumor biopsies on sequential CTLA-4 and PD-1 blockade reveals markers of response and resistance. Sci Transl Med 9 (2017).
26. W. N. William, Jr. et al., Erlotinib and the Risk of Oral Cancer: The Erlotinib Prevention of Oral Cancer (EPOC) Randomized Clinical Trial. JAMA Oncol 2, 209-216 (2016).
27. A. M. Goodman et al., Prevalence of PDL1 Amplification and Preliminary Response to Immune Checkpoint Blockade in Solid Tumors. JAMA Oncol 4, 1237-1244 (2018).
28. E. Becht et al., Estimating the population abundance of tissue-infiltrating immune and stromal cell populations using gene expression. Genome Biol 17, 218 (2016).
29. F. Finotello et al., Molecular and pharmacological modulators of the tumor immune contexture revealed by deconvolution of RNA-seq data. Genome Med 11, 34 (2019).
30. A. M. Newman et al., Determining cell type abundance and expression from bulk tissues with digital cytometry. Nat Biotechnol 37, 773-782 (2019).
31. D. Aran, Z. Hu, A. J. Butte, xCell: digitally portraying the tissue cellular heterogeneity landscape. Genome Biol 18, 220 (2017).
32. C. Huang et al., Proteogenomic insights into the biology and treatment of HPV-negative head and neck squamous cell carcinoma. Cancer Cell 39, 361-379.e316 (2021).
33. K. A. Hoadley et al., Multiplatform analysis of 12 cancer types reveals molecular classification within and across tissues of origin. Cell 158, 929-944 (2014).
34. A. M. Taylor et al., Genomic and Functional Approaches to Understanding Cancer Aneuploidy. Cancer Cell 33, 676-689 e673 (2018).
35. E. M. Ebot et al., Deletions on 9p21 are associated with worse outcomes after anti-PD-1/PD-L1 monotherapy but not chemoimmunotherapy. NPJ Precis Oncol 6, 44 (2022).
36. T. E. Keenan, K. P. Burke, E. M. Van Allen, Genomic correlates of response to immune checkpoint blockade. Nat Med 25, 389-402 (2019).
37. A. Prestipino et al., Oncogenic JAK2 (V617F) causes PD-L1 expression, mediating immune escape in myeloproliferative neoplasms. Sci Transl Med 10 (2018).
38. M. Chen et al., JAK2 and PD-L1 Amplification Enhance the Dynamic Expression of PD-L1 in Triplenegative Breast Cancer. Clin Breast Cancer 18, e1205-e1215 (2018).
39. V. Saloura et al., Immune profiles in primary squamous cell carcinoma of the head and neck. Oral Oncol 96, 77-88 (2019).
40. D. Tamborero et al., A Pan-cancer Landscape of Interactions between Solid Tumors and Infiltrating Immune Cell Populations. Clin Cancer Res 24, 3717-3728 (2018).
41. J. D. Campbell et al., Genomic, Pathway Network, and Immunologic Features Distinguishing Squamous Carcinomas. Cell Rep 23, 194-212.e196 (2018).
42. R. S. Herbst, J. V. Heymach, S. M. Lippman, Lung Cancer. New England Journal of Medicine 359, 1367-1380 (2008).
43. F. M. Barriga et al., Chromosome 9p21.3 coordinates cell intrinsic and extrinsic tumor suppression. Nature (in press).
44. C. H. Mermel et al., GISTIC2.0 facilitates sensitive and confident localization of the targets of focal somatic copy-number alteration in human cancers. Genome Biol 12, R41 (2011).

### REFERENCES FOR EXPERIMENT NO. 2

1. S. L. Carter et al., Absolute quantification of somatic DNA alterations in human cancer. Nat Biotechnol 30, 413-421 (2012).
2. T. Davoli, H. Uno, E. C. Wooten, S. J. Elledge, Tumor aneuploidy correlates with markers of immune evasion and with reduced response to immunotherapy. Science 355 (2017).
3. Y. Benjamini, Y. Hochberg, Controlling the False Discovery Rate: A Practical and Powerful Approach to Multiple Testing. Journal of the Royal Statistical Society: Series B (Methodological) 57, 289-300 (1995).
4. E. Becht et al., Estimating the population abundance of tissue-infiltrating immune and stromal cell populations using gene expression. Genome Biol 17, 218 (2016).
5. F. Finotello et al., Correction to: Molecular and pharmacological modulators of the tumor immune contexture revealed by deconvolution of RNA-seq data. Genome Med 11, 50 (2019).
6. A. M. Newman et al., Determining cell type abundance and expression from bulk tissues with digital cytometry. Nat Biotechnol 37, 773-782 (2019).
7. D. Aran, Z. Hu, A. J. Butte, xCell: digitally portraying the tissue cellular heterogeneity landscape. Genome Biol 18, 220 (2017).
8. J. E. Beane et al., Molecular subtyping reveals immune alterations associated with progression of bronchial premalignant lesions. Nat Commun 10, 1856 (2019).
9. E. Talevich, A. H. Shain, T. Botton, B. C. Bastian, CNVkit: Genome-Wide Copy Number Detection and Visualization from Targeted DNA Sequencing. PLOS Comput Biol 12, e1004873 (2016).
10. R. Patro, G. Duggal, M. I. Love, R. A. Irizarry, C. Kingsford, Salmon provides fast and bias-aware quantification of transcript expression. Nat Methods 14, 417-419 (2017).
11. J. Abraham et al., Machine learning analysis using 77,044 genomic and transcriptomic profiles to accurately predict tumor type. Translational Oncology 14, 101016 (2021).
12. K. Emancipator et al., Comparing programmed death ligand 1 scores for predicting pembrolizumab efficacy in head and neck cancer. Mod Pathol 34, 532-541 (2021).

1. A method of treating cancer in a subject in need thereof, comprising administering to the subject a treatment comprising at least one of Apitolisib, Torn 2, or GSK1059615 when a 9p gain is detected in a sample obtained from the subject.

2. The method of claim 1, wherein the cancer comprises human papilloma virus negative head and neck cancer subject.

3. The method of claim 1, wherein the sample comprises a cell line, optionally wherein the cell line comprises at least 1, at least 2, at least 3, or at least 4 cell lines.

4. The method of claim 1, wherein the measuring 9p gain of the sample comprises chromosomal microarrays configured to detect 9p gain of the sample, target panel sequencing of the sample, sequencing the sample's exome, sequencing the sample's whole genome, using a trained predictive model to analyze a structure of the sample stained with hematoxylin and eosin, or any combination thereof, and optionally wherein the trained predictive model is trained with a plurality of chromosome 9p classification features.

5. The method of claim 3, wherein the cell line comprises at least 1, at least 2, at least 3, or at least 4 cell lines.

6. (canceled)

7. The method of claim 4, wherein the sample's exome, whole genome, any fraction thereof, or any combination thereof are obtained from a publicly available database.

8. The method of claim 7, wherein the publicly available database comprises Genomic of Drug Sensitivity in Cancer (GDSC), Cancer Cell Line Encyclopedia (CCLE), cancer genome atlas program (TCGA), Real World Cohort (RWC), Clinical Proteomic Tumor Analysis Consortium (CPTAC), or any combination thereof.

9. The method of claim 1, wherein the treatment comprises at least about 10, at least about 20, at least about 23, or at least about 30-fold increase in IC50 value when administered to the subject with the 9p gain in the sample.

10. The method of claim 1, wherein the 9p gain comprises a 9p24.1 or a 9p21.3 gain.

11. The method of claim 1, wherein the measured 9p gain comprises a 9p24.1 expression threshold of at least a 60th percentile.

12. (canceled)

13. The method of claim 1, wherein the cancer comprises lung adenocarcinoma, lung squamous cell carcinoma, pancreatic adenocarcinoma, bladder urothelial carcinoma, skin cutaneous melanoma, ESCA-Squamous (esophageal carcinoma), ESCA-Adenocarcinoma (esophageal carcinoma), stomach adenocarcinoma, colorectal adenocarcinoma, cervical cancer, breast cancer, ovarian cancer, prostate cancer, sarcoma, or any combination thereof cancer.

14. A method of treating cancer in a subject in need thereof, comprising administering to the subject a treatment that targets PI3k, Akt, mTOR, STING agonist signaling pathways or any combination thereof when a 9p gain is detected in a sample obtained from the subject.

15. The method of claim 14, wherein the subject comprises a human papilloma virus negative head and neck cancer subject.

16. The method of claim 14, wherein the sample comprises a cell line, optionally wherein the cell line comprises at least 1, at least 2, at least 3, or at least 4 cell lines.

17. The method of claim 14, wherein the measuring 9p gain in the sample comprises chromosomal microarrays configured to detect 9p gain in the sample, target panel sequencing of the sample, sequencing the sample's exome, sequencing the sample's whole genome, using a trained predictive model to analyze a structure of the sample stained with hematoxylin and eosin, or any combination thereof.

18. (canceled)

19. The method of claim 17, wherein the trained predictive model is trained with a plurality of chromosome 9p classification features.

20. The method of claim 17, wherein the sample's exome, whole genome, any fraction thereof, or any combination thereof are obtained from a publicly available database.

21. The method of claim 20, wherein the publicly available database comprises Genomic of Drug Sensitivity in Cancer (GDSC), cancer genome atlas program (TCGA), Real World Cohort (RWC), Clinical Proteomic Tumor Analysis Consortium (CPTAC), Cancer Cell Line Encyclopedia (CCLE), or any combination thereof.

22. The method of claim 14, wherein the treatment comprises at least about 10, at least about 20, at least about 23, or at least about 30-fold increase in IC50 value when administered to the subject with the 9p gain in the sample.

23. The method of claim 14, wherein the 9p gain comprises a 9p24.1 or a 9p21.3 gain.

24. (canceled)

25. (canceled)

26. The method of claim 14, wherein the cancer comprises lung adenocarcinoma, lung squamous cell carcinoma, pancreatic adenocarcinoma, bladder urothelial carcinoma, skin cutaneous melanoma, ESCA-Squamous (esophageal carcinoma), ESCA-Adenocarcinoma (esophageal carcinoma), stomach adenocarcinoma, colorectal adenocarcinoma, cervical cancer, breast cancer, ovarian cancer, prostate cancer, sarcoma, or any combination thereof cancer.


SEQUENCE LISTING (2 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET